SEQUENCE LISTING (66 sequences)

SEQ ID NO: 1 - protein, 90 aa, Artificial Sequence
  Description of Artificial Sequence Synthetic polypeptide
  Recombinant protein
TEENVKRRTHNVLERQRRNELKRSFFALRDQIPELENNEKAPKVVILKKATAYILSVQAETQKLISEIDLLRKQNEQLKHKLEQLRNSCA

SEQ ID NO: 2 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 3 - protein, 11 aa, Artificial Sequence
  Description of Artificial Sequence Synthetic peptide
  Recombinant protein
RQRRNELKRSF

SEQ ID NO: 4 - protein, 91 aa, Artificial Sequence
  Description of Artificial Sequence Synthetic polypeptide
  Recombinant protein
MTEENVKRRTHNVLERQRRNELKRSFFALRDQIPELENNEKAPKVVILKKATAYILSVQAETQKLISEIDLLRKQNEQLKHKLEQLRNSCA

SEQ ID NO: 5 - protein, 90 aa, Artificial Sequence
  Description of Artificial Sequence Synthetic polypeptide
  Recombinant protein
  MOD_RES (89)..(89): Any amino acid except Cysteine
TEENVKRRTHNVLERQRRNELKRSFFALRDQIPELENNEKAPKVVILKKATAYILSVQAETQKLISEIDLLRKQNEQLKHKLEQLRNSXA

SEQ ID NO: 6 - protein, 91 aa, Artificial Sequence
  Description of Artificial Sequence Synthetic polypeptide
  Recombinant protein
  MOD_RES (90)..(90): Any amino acid except Cysteine
MTEENVKRRTHNVLERQRRNELKRSFFALRDQIPELENNEKAPKVVILKKATAYILSVQAETQKLISEIDLLRKQNEQLKHKLEQLRNSXA

SEQ ID NO: 7 - protein, 90 aa, Artificial Sequence
  Description of Artificial Sequence Synthetic polypeptide
  Recombinant protein
TEENVKRRTHNVLERQRRNELKRSFFALRDQIPELENNEKAPKVVILKKATAYILSVQAETQKLISEIDLLRKQNEQLKHKLEQLRNSSA

SEQ ID NO: 8 - protein, 91 aa, Artificial Sequence
  Description of Artificial Sequence Synthetic polypeptide
  Recombinant protein
MTEENVKRRTHNVLERQRRNELKRSFFALRDQIPELENNEKAPKVVILKKATAYILSVQAETQKLISEIDLLRKQNEQLKHKLEQLRNSSA

SEQ ID NO: 9 - protein, 90 aa, Artificial Sequence
  Description of Artificial Sequence Synthetic polypeptide
  Recombinant protein
TEENVKRRTHNVLERQRRNELKRSFFALRDQIPELENNEKAPKVVILKKATAYILSVQAETQKLISEIDLLRKQNEQLKHKLEQLRNSAA

SEQ ID NO: 10 - protein, 91 aa, Artificial Sequence
  Description of Artificial Sequence Synthetic polypeptide
  Recombinant protein
MTEENVKRRTHNVLERQRRNELKRSFFALRDQIPELENNEKAPKVVILKKATAYILSVQAETQKLISEIDLLRKQNEQLKHKLEQLRNSAA

SEQ ID NO: 11 - protein, 101 aa, Artificial Sequence
  Description of Artificial Sequence Synthetic polypeptide
  Recombinant protein
MTEENVKRRTHNVLERQRRNELKRSFFALRDQIPELENNEKAPKVVILKKATAYILSVQAETQKLISEIDLLRKQNEQLKHKLEQLRNSCAGRKKRRQRRR

SEQ ID NO: 12 - protein, 99 aa, Artificial Sequence
  Description of Artificial Sequence Synthetic polypeptide
  Recombinant protein
MTEENVKRRTHNVLERQRRNELKRSFFALRDQIPELENNEKAPKVVILKKATAYILSVQAETQKLISEIDLLRKQNEQLKHKLEQLRNSCARRRRRRLR

SEQ ID NO: 13 - protein, 16 aa, Artificial Sequence
  Description of Artificial Sequence Synthetic peptide
  CPP sequence found in Drosophila antennapedia protein
RQIKIWFQNRRMKWKK

SEQ ID NO: 14 - protein, 34 aa, Artificial Sequence
  Description of Artificial Sequence Synthetic polypeptide
  CPP sequence found in the herpesvirus simplex 1 (HSV-1) VP22 DNA-binding protein
DAATATRGRSAASRPTERPRAPARSASRPRRPVE

SEQ ID NO: 15 - protein, 24 aa, Artificial Sequence
  Description of Artificial Sequence Synthetic peptide
  CPP sequence of Bac-7
RRIRPRPPRLPRPRPRPLPFPRPG

SEQ ID NO: 16 - protein, 9 aa, Artificial Sequence
  Description of Artificial Sequence Synthetic peptide
  CPP sequence of the HIV-1 TAT protein (amino acids 49-57)
RKKRRQRRR

SEQ ID NO: 17 - protein, 13 aa, Artificial Sequence
  Description of Artificial Sequence Synthetic peptide
  CPP sequence of the HIV-1 TAT protein (amino acids 48-60)
GRKKRRQRRRTPQ

SEQ ID NO: 18 - protein, 11 aa, Artificial Sequence
  Description of Artificial Sequence Synthetic peptide
  CPP sequence of the HIV-1 TAT protein (amino acids 47-57)
YGRKKRRQRRR

SEQ ID NO: 19 - protein, 20 aa, Artificial Sequence
  Description of Artificial Sequence Synthetic peptide
  CPP sequence of S413-PV peptide
ALWKTLLKKVLKAPKKKRKV

SEQ ID NO: 20 - protein, 15 aa, Artificial Sequence
  Description of Artificial Sequence Synthetic peptide
  CPP sequence of penetratin
RQIKWFQNRRMKWKK

SEQ ID NO: 21 - protein, 18 aa, Artificial Sequence
  Description of Artificial Sequence Synthetic peptide
  CPP sequence of SynB1
RGGRLSYSRRRFSTSTGR

SEQ ID NO: 22 - protein, 10 aa, Artificial Sequence
  Description of Artificial Sequence Synthetic peptide
  CPP sequence of SynB3
RRLSYSRRRF

SEQ ID NO: 23 - protein, 12 aa, Artificial Sequence
  Description of Artificial Sequence Synthetic peptide
  CPP sequence of PTD-4
PIRRRKKLRRLK

SEQ ID NO: 24 - protein, 12 aa, Artificial Sequence
  Description of Artificial Sequence Synthetic peptide
  CPP sequence of PTD-5
RRQRRTSKLMKR

SEQ ID NO: 25 - protein, 15 aa, Artificial Sequence
  Description of Artificial Sequence Synthetic peptide
  CPP sequence of the FHV COat (amino acids 35- 49)
RRRRNRTRRNRRRVR

SEQ ID NO: 26 - protein, 19 aa, Artificial Sequence
  Description of Artificial Sequence Synthetic peptide
  CPP sequence of BMV Gag (amino acids 7-25)
KMTRAQRRAAARRNRWTAR

SEQ ID NO: 27 - protein, 13 aa, Artificial Sequence
  Description of Artificial Sequence Synthetic peptide
  CPP sequence of HTLV-II Rex (amino acids 4-16)
TRRQRTRRARRNR

SEQ ID NO: 28 - protein, 13 aa, Artificial Sequence
  Description of Artificial Sequence Synthetic peptide
  CPP sequence of D-Tat
GRKKRRQRRRPPQ

SEQ ID NO: 29 - protein, 13 aa, Artificial Sequence
  Description of Artificial Sequence Synthetic peptide
  CPP sequence of R9-Tat
GRRRRRRRRRPPQ

SEQ ID NO: 30 - protein, 17 aa, Artificial Sequence
  Description of Artificial Sequence Synthetic peptide
  CPP sequence of MAP
KLALKLALKLALALKLA

SEQ ID NO: 31 - protein, 27 aa, Artificial Sequence
  Description of Artificial Sequence Synthetic peptide
  CPP sequence of SBP
MGLGLHLLVLAAALQGAWSQPKKKRKV

SEQ ID NO: 32 - protein, 27 aa, Artificial Sequence
  Description of Artificial Sequence Synthetic peptide
  CPP sequence of FBP
GALFLGWLGAAGSTMGAWSQPKKKRKV

SEQ ID NO: 33 - protein, 27 aa, Artificial Sequence
  Description of Artificial Sequence Synthetic peptide
  CPP sequence of MPG
GALFLGFLGAAGSTMGAWSQPKKKRKV

SEQ ID NO: 34 - protein, 27 aa, Artificial Sequence
  Description of Artificial Sequence Synthetic peptide
  CPP sequence of MPG(ENLS)
GALFLGFLGAAGSTMGAWSQPKSKRKV

SEQ ID NO: 35 - protein, 21 aa, Artificial Sequence
  Description of Artificial Sequence Synthetic peptide
  CPP sequence of Pep-1
KETWWETWWTEWSQPKKKRKV

SEQ ID NO: 36 - protein, 21 aa, Artificial Sequence
  Description of Artificial Sequence Synthetic peptide
  CPP sequence of Pep-2
KETWFETWFTEWSQPKKKRKV

SEQ ID NO: 37 - protein, 10 aa, Artificial Sequence
  Description of Artificial Sequence Synthetic peptide
  CPP sequence
GRKKRRQRRR

SEQ ID NO: 38 - protein, 8 aa, Artificial Sequence
  Description of Artificial Sequence Synthetic peptide
  CPP sequence
RRRRRRLR

SEQ ID NO: 39 - protein, 12 aa, Artificial Sequence
  Description of Artificial Sequence Synthetic peptide
  CPP sequence
RRQRRTSKLMKR

SEQ ID NO: 40 - protein, 27 aa, Artificial Sequence
  Description of Artificial Sequence Synthetic peptide
  Transportan peptide
GWTLNSAGYLLGKINLKALAALAKKIL

SEQ ID NO: 41 - protein, 33 aa, Artificial Sequence
  Description of Artificial Sequence Synthetic polypeptide
  CPP sequence
KALAWEAKLAKALAKALAKHLAKALAKALKCEA

SEQ ID NO: 42 - protein, 16 aa, Artificial Sequence
  Description of Artificial Sequence Synthetic peptide
  CPP sequence
RQIKIWFQNRRMKWKK

SEQ ID NO: 43 - protein, 11 aa, Artificial Sequence
  Description of Artificial Sequence Synthetic peptide
  CPP sequence
YGRKKRRQRRR

SEQ ID NO: 44 - protein, 8 aa, Artificial Sequence
  Description of Artificial Sequence Synthetic peptide
  CPP sequence
RKKRRQRR

SEQ ID NO: 45 - protein, 11 aa, Artificial Sequence
  Description of Artificial Sequence Synthetic peptide
  CPP sequence
YARAAARQARA

SEQ ID NO: 46 - protein, 11 aa, Artificial Sequence
  Description of Artificial Sequence Synthetic peptide
  CPP sequence
THRLPRRRRRR

SEQ ID NO: 47 - protein, 11 aa, Artificial Sequence
  Description of Artificial Sequence Synthetic peptide
  CPP sequence
GGRRARRRRRR

SEQ ID NO: 48 - protein, 7 aa, Artificial Sequence
  Description of Artificial Sequence Synthetic peptide
  NLS sequence of SV40 large T Antigen
PKKKRKV

SEQ ID NO: 49 - protein, 16 aa, Artificial Sequence
  Description of Artificial Sequence Synthetic peptide
  NLS sequence of Nucleoplasmin
KRPAATKKAGQAKKKK

SEQ ID NO: 50 - protein, 18 aa, Artificial Sequence
  Description of Artificial Sequence Synthetic peptide
  NLS sequence of CBP80
RRRHSDENDGGQPHKRRK

SEQ ID NO: 51 - protein, 11 aa, Artificial Sequence
  Description of Artificial Sequence Synthetic peptide
  NLS sequence of HIV-I Rev protein
RQARRNRRRWE

SEQ ID NO: 52 - protein, 18 aa, Artificial Sequence
  Description of Artificial Sequence Synthetic peptide
  NLS sequence of HTLV-I Rex
MPKTRRRPRRSQRKRPPT

SEQ ID NO: 53 - protein, 37 aa, Artificial Sequence
  Description of Artificial Sequence Synthetic polypeptide
  NLS of hnRNP A
NQSSNFGPMKGGNFGGRSSGPYGGGGQYFKPRNQGGY

SEQ ID NO: 54 - protein, 43 aa, Artificial Sequence
  Description of Artificial Sequence Synthetic polypeptide
  NLS of rpL23a
VHSHKKKKIRTSPTFTTPKTLRLRRQPKYPRKSAPRRNKLDHY

SEQ ID NO: 55 - protein, 4 aa, Artificial Sequence
  Description of Artificial Sequence Synthetic peptide
  NLS sequence
  MOD_RES (2)..(2): R or K
  MOD_RES (3)..(3): Any naturally occurring amino acid
  MOD_RES (4)..(4): R or K
KXXX

SEQ ID NO: 56 - protein, 9 aa, Artificial Sequence
  Description of Artificial Sequence Synthetic peptide
  M1 peptide (Myc NLS)
PAAKRVKLD

SEQ ID NO: 57 - protein, 11 aa, Artificial Sequence
  Description of Artificial Sequence Synthetic peptide
  M2 peptide (Myc NLS)
RQRRNELKRSF

SEQ ID NO: 58 - protein, 6 aa, Artificial Sequence
  Description of Artificial Sequence Synthetic peptide
  flexible peptide
GPRRRR

SEQ ID NO: 59 - protein, 8 aa, Artificial Sequence
  Description of Artificial Sequence Synthetic peptide
  FLAG-tag
DYKDDDDK

SEQ ID NO: 60 - protein, 8 aa, Artificial Sequence
  Description of Artificial Sequence Synthetic peptide
  Strep-tag
WSHPQFEK

SEQ ID NO: 61 - protein, 9 aa, Artificial Sequence
  Description of Artificial Sequence Synthetic peptide
  HA-tag
YPYDVPDYA

SEQ ID NO: 62 - protein, 14 aa, Artificial Sequence
  Description of Artificial Sequence Synthetic peptide
  V5 tag
GKPIPNPLLGLDST

SEQ ID NO: 63 - protein, 6 aa, Artificial Sequence
  Description of Artificial Sequence Synthetic peptide
  Peptide tag
AHGHRP

SEQ ID NO: 64 - protein, 21 aa, Artificial Sequence
  Description of Artificial Sequence Synthetic peptide
  Peptide tag
  MOD_RES (19)..(20): Any naturally occurring amino acid
PIHDHDHPHLVIHSGMTCXXC

SEQ ID NO: 65 - protein, 8 aa, Artificial Sequence
  Description of Artificial Sequence Synthetic peptide
  CPP
RRRRRRRR

SEQ ID NO: 66 - protein, 6 aa, Artificial Sequence
  Description of Artificial Sequence Synthetic 6xHis tag
HHHHHH

(19) United States
(12) Patent Application Publication
(10) Pub. No.: US 2023/0055103 A1
(43) Pub. Date: Feb. 23, 2023

(54) METHODS FOR THE DIAGNOSIS OF LUNG CANCER

(71) Applicants: FUNDACIÓ PRIVADA INSTITUT D'INVESTIGACIÓ ONCOLÒGICA DE VALL HEBRON, Barcelona (ES); INSTITUCIÓ CATALANA DE RECERCA | ESTUDIS AVANÇATS, Barcelona (ES); PEPTOMYC, S.L., Barcelona (ES)

(72) Inventors: Laura SOUCEK, Barcelona (ES); Marie-Eve BEAULIEU, Barcelona (ES)

(21) Appl. No.: 17/593,403

(22) PCT Filed: Mar. 19, 2020
(86) PCT No.: PCT/EP2020/057585

§ 371 Date: Sep. 17, 2021

(30) Foreign Application Priority Data
Mar. 19, 2019 (EP) 19382195.6

(51) Int. Cl. A61K 51/08; A61K 9/00

(52) U.S. Cl. CPC A61K 51/088; A61K 9/0043

(57) ABSTRACT

The invention relates to the diagnostic uses of a conjugate comprising Omomyc or a functionally equivalent variant thereof and a detectable label for detecting lung cancer by pulmonary administration of the conjugate. The invention also relates to a method for detecting or imaging lung cancer using said conjugates, kits comprising said conjugates and conjugates comprising a contrast agent or an imaging agent.

### CROSS-REFERENCE TO RELATED APPLICATIONS

The present application is a § 371 National Stage of PCT International Application No. PCT/EP2020/057585, filed Mar. 19, 2020, which claims the benefit of EP Patent Application No. EP19382195.6, filed Mar. 19, 2019, the entirety of each of which is hereby incorporated by reference.

### SEQUENCE LISTING

The instant application contains a Sequence Listing which has been submitted electronically in ASCII format and is hereby incorporated by reference in its entirety. Said ASCII copy, created on Apr. 13, 2022, is named 397724-004US1_185554_SL.txt and is 32,353 bytes in size.

### FIELD OF THE INVENTION

The present invention is comprised within the field of diagnosis, more specifically, within the field of diagnosis of lung cancer in vivo by means of a traceable agent which accumulates specifically into proliferative cells.

### BACKGROUND OF THE INVENTION

Lung cancer is one among cancers with high lethality all over the world as well as domestically, and such a trend is caused by absence of symptoms and early diagnosis methods having high sensitivity.

Most patients had proceeded to an advanced stage before diagnosis, and thus fail to be treated at an earlier stage. Two common types of lung cancer are small cell lung cancer (SCLC) (16.8%) and non-small cell lung cancer (NSCLC) (80.4%). Non-small cell lung cancer mainly includes squamous cell carcinoma, pulmonary adenocarcinoma and large cell lung cancer, among which pulmonary adenocarcinoma is the most common lung cancer (30%-65%). The pathogeny of lung cancer is not yet known.

Current medical studies have focused on the diagnosis and treatment of lung cancer at earlier stage. Statistically, non-small cell lung cancer patients at an earlier stage have a 5-year survival of up to 80%, while the total 5-year survival for non-small cell lung cancer is only 15%. Thus, it is important to diagnose and treat lung cancer at an earlier stage. Current methods for the diagnosis of lung cancer include sputum cytology, image testing, endoscopy, and biopsy. The sensitivity of sputum cytology is low. Imaging test methods commonly used for lung cancer include X-ray, CT, MRI (magnetic resonance imaging), ultrasound, nuclide imaging, PET-CT (positron emission tomography/computed tomography), and the like. Imaging test methods are not sensitive enough, and usually only a lesion more than 1 cm in size is visible. In endoscopy, a tumor is visible only when it resides in airway accessible to an endoscope. Low-dose chest CT has limited sensitivity, although it is the most recognized diagnosis method. Like X-ray testing, CT involves ionizing radiation which itself can lead to cancer.

The diagnostic rate for earlier-stage patients not displaying typical symptoms is only 15%. Traditional methods for screening lung cancer are lacking for high-risk populations due to their limited specificity and sensitivity, onerous nature, and high cost. These traditional methods fail to significantly decrease the mortality of lung cancer. Thus, there is a need for an alternative or supplemental method for screening lung cancer to increase diagnostic rate for early lung cancer, and reduce the number of surgical operations to lower the risk of complications.

### BRIEF SUMMARY OF THE INVENTION

In a first aspect, the invention relates to a conjugate comprising:
i) a polypeptide comprising the sequence SEQ ID NO: 1 or a functionally equivalent variant thereof, and
ii) a detectable label,
   for use in a method of diagnosis “in vivo” of lung cancer in a subject in need thereof by pulmonary administration of the conjugate.

In a second aspect, the invention relates to a method for detecting or imaging lung cancer cells in a subject comprising:
i) administering intranasally a conjugate comprising a polypeptide comprising the sequence SEQ ID NO: 1 or a functionally equivalent variant thereof, and a detectable label;
ii) waiting a sufficient time to allow for the conjugate to enter into the proliferating lung cells; and
iii) detecting the proliferating lung cells by applying an imaging technique to the subject to detect the site of accumulation of the conjugate in the subject, thereby detecting or imaging the site of proliferation.

In a third aspect, the invention relates to a kit for the diagnosis of lung cancer comprising:
i) a conjugate comprising a polypeptide comprising the sequence SEQ ID NO: 1 or a functionally equivalent variant thereof, and a detectable label,
ii) a device for the nasal instillation or nasal inhalation of the conjugate of item i); and
iii) means for packaging items i) and ii).

In a fourth aspect, the invention relates to the use of a kit according to the third aspect of the invention for diagnosing lung cancer or for monitoring the progression of lung cancer or for monitoring the effect of a therapy.

In a fifth aspect, the invention relates to a conjugate comprising:
i) a polypeptide comprising the sequence SEQ ID NO: 1 or a functionally equivalent variant thereof, and
ii) a detectable label selected from the group consisting of a contrast agent or an imaging agent.

### DESCRIPTION OF THE FIGURES

FIG. 1. Omomyc distributes to the lung in a KRas^{G12D}-driven lung adenocarcinoma mouse model upon intranasal administration. (A) Quantification of Omomyc-DFO-⁸⁹Zr detected in the lungs of healthy mice as a function of time is represented as % injected dose. Mean, S.D. and number of animals are shown. (B) Immunofluorescence of lung tissue from mice treated with the Omomyc mini-protein. A specific anti-Omomyc antibody confirms the presence of Omomyc in the lung cells 4 h after administration. Arrowheads indicate positively stained nuclei. Scale bar, 10 μm. Higher magnification of the area surrounded by a white-dashed line is shown in the right panel. Numbers correspond to corrected total cell fluorescence calculated by ImageJ (C) 3D rendering of microPET/CT imaging of lungs of a tumor-bearing mouse 24 h after intranasal administration of 2.37 mg/kg Omomyc-DFO-⁸⁹Zr. CT data are displayed in grey scale and Omomyc-DFO-⁸⁹Zr microPET data in color scale (n=2 mice were analyzed). Numbers correspond to % intranasal dose (ID)/g for Omomyc-DFO-89Zr uptake.

FIG. 2. Biodistribution and pharmacokinetic study. 2 mg/kg of Omomyc-DFO-⁸⁹Zr were administered intranasally to healthy control and to tumor-bearing Kras^{G12D} mice. (A) Lung image obtained 24 h after intranasal administration to tumor-bearing mice. Numbers correspond to % ID/g for Omomyc-DFO-89Zr uptake (B) Lung image obtained 24 h after intranasal administration to healthy controls. (C) Biodistribution from ex vivo quantification of radioactivity from Omomyc-DFO-⁸⁹Zr at each point following intranasal administration to mice with established lung adenocarcinoma.

FIG. 3. (A). Ex vivo fluorescence intensity of lung treated with OmomycCPP-AF660, 4 h after intranasal administration (1.4 mg/kg in 30 μL vehicle 10 mM sodium acetate pH 6.5). Scale bar, 1 cm. (B). 24 h after a single administration of OmomycCPP-AF660, the polypeptide remains detectable in lung tumors (FLI, Fluorescence Intensity). Bottom panel shows the same lungs where the macroscopically visible superficial tumors are indicated by circles. Scale bar, 1 cm.

FIG. 4. Biodistribution from ex vivo quantification of radioactivity at 72 hours after intravenous administration of 2.68 mg/kg of Omomyc-DFO-⁸⁹Zr. % of injected dose is shown per gram of indicated tissue.

### DETAILED DESCRIPTION OF THE INVENTION

The present invention relates to the provision of new agents for the diagnosis and/or monitoring of lung cancer.

Unless otherwise defined, all technical terms used herein have the same meaning as commonly understood by one of ordinary skill in the art to which this invention belongs.

All the embodiments disclosed in the context of one aspect of the invention are also applicable to the other aspects of the invention.

Diagnostic Uses of the Conjugates of the Invention

The definitions provided herewith and in every other aspect of the invention are equally applicable to the whole invention.

As disclosed in the examples, the inventors of the present invention have demonstrated that a conjugate comprising the polypeptide of SEQ ID NO: 1 and a detectable label such as a fluorescent label (AF660) or a radioisotope label (⁸⁹Zr) can be used as a lung tumor tracer when administered in vivo by intranasal route, since it specifically accumulates into cancerous cells being able to discriminate between healthy and proliferative cells. Surprisingly, the conjugate of the invention accumulated in the tumors by 24 hours whereas it was washed off from the normal tissue.

Thus, in a first aspect the invention relates to a conjugate comprising:
i) a polypeptide comprising the sequence SEQ ID NO: 1 or a functionally equivalent variant thereof, and
ii) a detectable label,
   for use in a method of diagnosis “in vivo” of lung cancer in a subject in need thereof by pulmonary administration of the conjugate.

In another aspect the invention relates to the use of a conjugate comprising:
i) a polypeptide comprising the sequence SEQ ID NO: 1 or a functionally equivalent variant thereof, and
ii) a detectable label,
   for diagnosing or detecting lung cancer in a subject by pulmonary administration of the conjugate.

The term “conjugate”, as used herein, refers to two or more compounds which are bound together so that the function of each compound is retained in the conjugate. The bonding of the two compounds may be a physical or chemical interaction, preferably a chemical interaction, more preferably an ionic or covalent bond; more preferably a covalent bond.

The terms “polypeptide” and “peptide” are used interchangeably herein to refer to polymers of amino acids of any length. The polypeptide of the invention can comprise modified amino acids, and it can be interrupted by non-amino acids. In a preferred embodiment the polypeptide is exclusively formed by amino acids. Preferably the polypeptide that forms item (i) of the conjugate has a length between 80 and 500 amino acids, more preferable between 80 and 300 amino acids, more preferable between 80 and 250 amino acids, more preferably between 80 and 150, even more preferably between 80 and 130 amino acids, preferably between 90 and 130 amino acids, preferably no more than 125 amino acids, more preferably no more than 100 amino acids. In a preferred embodiment, the polypeptide has a length between 90 and 98 amino acids, preferably between 90 and 95 amino acids, more preferably 91 amino acids.

The term “amino acid” refers to naturally occurring amino acids and non-natural (synthetic) amino acids, as well as amino acid analogs and amino acid mimetics that function in a manner similar to the naturally occurring amino acids. Furthermore, the term “amino acid” includes both D- and L-amino acids (stereoisomers), preferably L-amino acids.

The term “natural amino acids” or “naturally occurring amino acids” comprises the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine.

As used herein, the term “non-natural amino acid” or “synthetic amino acid” refers to a carboxylic acid, or a derivative thereof, substituted at position “a” with an amine group and being structurally related to a natural amino acid. Illustrative, non-limiting examples of modified or uncommon amino acids include 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxy lysine, alio hydroxy lysine, 3-hydroxyproline, 4-hydroxyproline, isodesmosine, alloisoleucine, N-methylglycine, N-methyliso leucine, 6-N-methyl-lysine, N-methylvaline, norvaline, norleucine, ornithine, etc.

The polypeptide of the present invention may also comprise non-amino acid moieties, such as for example, hydrophobic moieties (various linear, branched, cyclic, polycyclic or heterocyclic hydrocarbons and hydrocarbon derivatives) attached to the peptides; various protecting groups which are attached to the compound's terminals to decrease degradation. Suitable protecting functional groups are described in Green and Wuts, “Protecting Groups in Organic Synthesis”, John Wiley and Sons, Chapters 5 and 7, 1991.

Chemical (non-amino acid) groups present in the polypeptide may be included in order to improve various physiological properties such; decreased degradation or clearance; decreased repulsion by various cellular pumps, improve various modes of administration, increased specificity, increased affinity, increased stability, bioavailability, solubility, decreased toxicity and the like.

“Mimetic” include molecules which mimic the chemical structure of a peptidic structure and retain the functional properties of the peptidic structure. Approaches to designing peptide analogs, derivatives and mimetics are known in the art.

In an embodiment component (i) of the conjugate is a polypeptide consisting of the sequence SEQ ID NO: 1 or a polypeptide consisting of a functionally equivalent variant of SEQ ID NO: 1, preferably is a polypeptide consisting of the sequence SEQ ID NO: 1.

The SEQ ID NO: 1 corresponds to

(SEQ ID NO: 1)
TEENVKRRTHNVLERQRRNELKRSFFALRDQIPELENNEKAPKVVILK
KATAYILSVQAETQKLISEIDLLRKQNEQLKHKLEQLRNSCA

The polypeptide of sequence SEQ ID NO: 1 corresponds to the Omomyc protein sequence. The term “Omomyc”, as used herein, refers to a polypeptide which consists of a mutated version of the bHLHZip domain of the Myc protein carrying the E61T, E681, R74Q and R75N mutations (wherein the numbering of the mutated positions is given with respect to the sequence of Myc region corresponding to amino acids 365-454 of the polypeptide as defined under accession number NP_002458 in the NCBI database, release of Mar. 15, 2015). The sequence of c-Myc provided in the NCBI database under the accession number NP_002458 is shown below (SEQ ID NO: 2), wherein the region from which Omomyc derives is shown underlined:

| | |
|---|---|
| 1 | MDFFRVVENQ QPPATMPLNV SFTNRNYDLD YDSVQPYFYC DEEENFYQQQ QQSELQPPAP |
| 61 | SEDIWKKFEL LPTPPLSPSR RSGLCSPSYV AVTPFSLRGD NDGGGGSFST ADQLEMVTEL |
| 121 | LGGDMVNQSF ICDPDDETFI KNIIIQDCMW SGFSAAAKLV SEKLASYQAA RKDSGSPNPA |
| 181 | RGHSVCSTSS LYLQDLSAAA SECIDPSVVF PYPLNDSSSP KSCASQDSSA FSPSSDSLLS |
| 241 | STESSPQGSP EPLVLHEETP PTTSSDSEEE QEDEEEIDVV SVEKRQAPGK RSESGSPSAG |
| 301 | GHSKPPHSPL VLKRCHVSTH QHNYAAPPST RKDYPAAKRV KLDSVRVLRQ ISNNRKCTSP |
| 361 | RSSDTEENVK RRTHNVLE [symbol] [symbol] F ALRDQIPELE NNEKAPKVVI LKKATAYILS |
| 421 | VQAEEQKLIS EEDLLRKRRE QLKHKLEQLR NSCA (SEQ ID NO: 2) |

Omomyc also contains the M2 domain of c-Myc, having the sequence RQRRNELKRSF (SEQ ID NO: 3) (see Dang and Lee, Mol. Cell. Biol., 1988, 8:4048-4054) (double underlined above), and which corresponds to a nuclear localization signal.

Omomyc is characterized in that it shows increased dimerization capacity with all three oncogenic Myc proteins (c-Myc, N-Myc and L-Myc). Omomyc can derive from the bHLHZip domain of any Myc protein known in the art, provided that the mutations which result in the tumor suppressor effect are preserved. Thus, the Omomyc that can be used in the present invention may derive from any mammal species, including but not being limited to domestic and farm animals (cows, horses, pigs, sheep, goats, dog, cats or rodents), primates and humans. Preferably, the Omomyc protein is derived from human Myc protein (accession number NP_002458, release of Mar. 12, 2019).

The term “Myc”, as used herein, refers to a family of transcription factors which includes c-Myc, N-Myc and L-Myc. Myc protein activates expression of many genes through binding on consensus sequence CACGTG (Enhancer Box sequences or E-boxes and recruiting histone acetyl-transferases or HATs). However, Myc can also act as a transcriptional repressor. By binding the Miz-1 transcription factor and displacing p300 co-activator, it inhibits expression of Miz-1 target genes. Myc also has a direct role in the control of DNA replication.

The Myc b-HLH-LZ or Myc basic region helix-loop-helix leucine zipper domain refers to a region which determines Myc dimerization with Max protein and binding to Myc-target genes. This region corresponds to amino acids 365-454 of human Myc and is characterized by two alpha helices connected by a loop (Nair, S. K., & Burley, S. K., 2003, Cell, 112: 193-205).

In a preferred embodiment, component (i) of the conjugate is a polypeptide that comprises, consists of or consists essentially of the SEQ ID NO: 4 shown below.

(SEQ ID NO: 4)
MTEENVKRRTHNVLERQRRNELKRSFFALRDQIPELENNEKAPKVVILK
KATAYILSVQAETQKLISEIDLLRKQNEQLKHKLEQLRNSCA

In this context, “consisting essentially of” means that the specified molecule would not contain any additional sequences that would alter the activity of SEQ ID NO: 4.

Preferably, the polypeptide consists of SEQ ID NO: 4.

The term “functionally equivalent variant”, refers to any polypeptide which results from the insertion or addition of one or more amino acids and/or from the deletion of one or more amino acids and/or from the conservative substitution of one or more amino acids with respect to the polypeptide of SEQ ID NO: 1 and/or which results from the chemical modification of the polypeptide of SEQ ID NO: 1 and which substantially preserves the tumor tracing activity of the SEQ ID NO: 1. Preferably, the functionally equivalent variant refers to any polypeptide which results from the insertion or addition of one or more amino acids and/or from the deletion of one or more amino acids and/or from the conservative substitution of one or more amino acids with respect to the polypeptide of SEQ ID NO: 1 and which substantially preserves the tumor tracing activity of SEQ ID NO: 1; more preferably results from the insertion or addition of one or more amino acids with respect to the polypeptide of SEQ ID NO: 1.

The skilled person will understand that the preservation of the tumor tracing activity requires that the variant is capable of penetrating into the cell. Therefore, functionally equivalent variants of Omomyc are capable of translocating across the cell membrane. Functionally equivalent variants of Omomyc are capable of transducing cells after the variant is contacted with said cell. It will be understood that functionally equivalent variants of Omomyc contain the protein transducing domain found in native Omomyc or another functional protein transducing domain. Therefore, in a preferred embodiment, the functionally equivalent variants of Omomyc are capable of translocating across the cell membrane.

In a preferred embodiment, a polypeptide is considered as a functionally equivalent variant of SEQ ID NO: 1 if it is capable of transducing a target cell at least 10%, 20%, 30%, 40%, 50%, 60%, 70%, 80%, 90% or 100% as efficiently as SEQ ID NO: 1.

Suitable assays for determining whether a polypeptide is a functionally equivalent variant of SEQ ID NO: 1 in terms of its ability to translocate across the cellular membrane include labelling of a cell with a reagent specific for the polypeptide. The detection of the polypeptide of the invention can be performed by confocal microscopy, flow citometry or by fluorescence microscopy assays using Omomyc specific antibodies or Omomyc labelled with suitable fluorophores. The detection can also be performed by cell fraction autoradiography assays to identify radiolabelled Omomyc.

Additionally, functionally equivalent variants of SEQ ID NO: 1 may also be capable of translocating across the nuclear envelope. In an embodiment, the functionally equivalent variant of Omomyc requires to be capable of translocating across the nuclear envelope. In another embodiment, the functionally equivalent variant of Omomyc does not require to be capable of translocating across the nuclear envelope.

In a preferred embodiment, a polypeptide is considered as a functionally equivalent variant of SEQ ID NO: 1 if it is capable of translocating to the nucleus of the target tumor cells at least 10%, 20%, 30%, 40%, 50%, 60%, 70%, 80%, 90% or 100% as efficiently as the SEQ ID NO: 1.

Suitable assays for determining whether a polypeptide is a functionally equivalent variant in terms of its ability to translocate to the nucleus include double labelling of a cell with a reagent specific for the polypeptide as disclosed above and with a dye which specifically labels the nucleus of the cell (such as DAPI or Hoechst dye). The detection of the polypeptide of the invention can be performed by confocal microscopy, flow citometry or by fluorescence microscopy.

The preservation of the tumor tracing activity may require that the variant can dimerize with Myc and/or its obligate partner p21/p22Max and inhibit Myc activity. In an embodiment, the functionally equivalent variant of Omomyc does not require dimerization with Myc and/or its obligate partner p21/p22 Max and inhibit Myc activity in order to preserve the tumor tracing activity. In another embodiment, the functionally equivalent variant of Omomyc requires that the variant can dimerize with Myc and/or its obligate partner p21/p22Max and inhibit Myc activity.

In some embodiments, the functionally equivalent variant of the polypeptide of the invention homodimerizes less than Omomyc, or is not forced into homodimers by the formation of disulphide bridge.

“Less homodimerization”, as used herein, relates to the lower ability of forming obligate homodimers of the polypeptide of the invention even in reducing conditions. In a preferred embodiment, the ability is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% less than the ability of forming homodimers of Omomyc. Reducing conditions, as used herein, relates to the presence of a reducing agent, a compound that donates an electron to another chemical species in a redox chemical reaction. Illustrative, non-limitative examples of reducing agents are DTT (dithiothreitol), b-mercaptoethanol or TCEP (tris(2-carboxyethyl)phosphine). It is possible that the amount of homodimers is the same in vitro, and that the difference between the functionally equivalent variant and Omomyc is present only in cells in presence of heterodimerization partners where the absence of the disulfide enables a potentially higher formation of heterodimers.

Several assays may be used to determine the homodimerization of a peptide, by way of illustrative non-limitative example by thermal denaturation monitored by Circular dichroism, so dimerization may be detected through folding and thermal stability quantification.

Suitable functionally equivalent variants include polypeptides consisting essentially of the polypeptide of SEQ ID NO: 1. In this context, “consisting essentially of” means that the specified molecule would not contain any additional sequences that would alter the activity of the SEQ ID NO: 1.

In a preferred embodiment, the functionally equivalent variant of SEQ ID NO: 1 is a polypeptide which results from the insertion or addition of one or more amino acids with respect to the polypeptide of SEQ ID NO: 1. In an embodiment, the functionally equivalent variant results from the insertion of less than 10 amino acids, more preferably less than 5 amino acids, more preferably results from the insertion of one amino acid. In a preferred embodiment, results from the insertion of one amino acid that is methionine.

In another embodiment, the functionally equivalent variant of SEQ ID NO: 1 is a polypeptide which results from the deletion of one or more amino acids with respect to the polypeptide of SEQ ID NO: 1. In an embodiment, the functionally equivalent variant results from the deletion of less than 10 amino acids, more preferably less than 5 amino acids, more preferably results from the deletion of one amino acid.

Suitable functional variants of the targeting peptide are those showing a degree of identity with respect to the peptide of SEQ ID NO:1 of about greater than 25% amino acid sequence identity, such as 25%, 30%, 40%, 50%, 60%, 65%, 70%, 75%, 80%, 85%, 90%, 91%, 92%, 93%, 94%, 95%, 96%, 97%, 98% or 99%. The degree of identity between two polypeptides is determined using computer algorithms and methods that are widely known for the persons skilled in the art. The identity between two amino acid sequences is preferably determined by using the BLASTP algorithm as described previously [BLAST Manual, Altschul, S., et al., NCBI NLM NIH Bethesda, Md. 20894, Altschul, S., et al., J. Mol. Biol. 1990; 215: 403-410]. In a preferred embodiment, the sequence identity is determined throughout the whole length of the polypeptide of SEQ ID NO: 1 or throughout the whole length of the variant or of both.

The functionally equivalent variants of the polypeptide of the invention may also include post-translational modifications, such as glycosylation, acetylation, isoprenylation, myristoylation, proteolytic processing, etc.

Suitable functional variants of the targeting peptide are those wherein one or more positions within the polypeptide of the invention contain an amino acid which is a conservative substitution of the amino acid present in the sequence mentioned above. “Conservative amino acid substitutions” result from replacing one amino acid with another having similar structural and/or chemical properties. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); and 6) Phenylalanine (F), Tyrosine (Y), Tryptophan (W). Selection of such conservative amino acid substitutions is within the skill of one of ordinary skill in the art and is described, for example, by Dordo et al., (J. Mol. Biol, 1999, 217; 721-739) and Taylor et al., (J. Theor. Biol., 1986, 119:205-218).

It will be understood that the functionally equivalent variants of Omomyc contain mutations at positions corresponding to the mutations E61T, E681, R74Q and R75N found in Omomyc derived from human c-Myc. The position wherein said mutations have to occur in the functionally equivalent variant can be determined by a multiple sequence alignment of different Myc sequences and identified by the alignment of those positions corresponding to positions 61, 68, 74 and 75 within the sequence of Omomyc derived from human c-Myc. In an embodiment, the functionally equivalent variants of Omomyc contain mutations at positions corresponding to the mutations E61T, E681, R74Q and R75N found in Omomyc derived from human c-Myc.

In another embodiment, the functionally equivalent variants of Omomyc contain mutations at positions corresponding to E61, E68, R74 and R75 within the sequence of Omomyc wherein E61 has been mutated to E61A or E61S; E68 has been mutated to E68L, E68M or E68V; R74 has been mutated to R74N; and R75 has been mutated to R75Q.

A multiple sequence alignment is an extension of pairwise alignment to incorporate more than two sequences at a time. Multiple alignment methods align all of the sequences in a given query set. A preferred multiple sequence alignment program (and its algorithm) is ClustalW, Clusal2W or ClustalW XXL (see Thompson et al. (1994) Nucleic Acids Res 22:4673-4680). Once the sequences of c-Myc from different organisms and of the variant are compared (aligned) as described herein, the skilled artisan can readily identify the positions within each of the sequence corresponding to positions E61T, E681, R74Q and R75N found in Omomyc and introduce within the Omomyc variant mutations corresponding to the E61T, E681, R74Q and R75N mutations found in Omomyc derived from human c-Myc.

In a preferred embodiment, a functionally equivalent variant of SEQ ID NO: 1 include those sequences having one or more, preferably all the following features: ability to dimerize with Myc and inhibiting its activity, translocation across the cell membrane, translocation to the nucleus, inability to form homodimers or reduced capacity to form homodimers compared to Omomyc.

Suitable assays for determining whether a polypeptide can be considered as a functionally equivalent variant of Omomyc include, without limitation:
Assays which measure the capacity of the polypeptide to form dimeric complexes with Max and Myc, such as the assays based on the expression of a reporter gene as described in Soucek et al. (Oncogene, 1998, 17: 2463-2472) as well as PLA (protein Ligation assay) or Co-immunoprecipitation.
Assays which measure the capacity of the polypeptide to bind to the Myc/Max recognition site within DNA (the CACGTG site), such as the electrophoretic mobility shift assay (EMSA) described in Soucek et al. (supra.)
Assays which measure the capacity to repress Myc-induced transactivation, such as the assay based on the expression of a reporter gene under the control of the DNA binding sites specific for Myc/Max as described by Soucek et al. (supra.).
Assays based on the capacity of the polypeptide to inhibit growth of cells expressing the myc oncogene, as described by Soucek et al. (supra.).
Assays which measure the ability of the polypeptide to enhance myc-induced apoptosis, such as the assays described by Soucek et al. (Oncogene, 1998: 17, 2463-2472). Moreover, any assay commonly known in the art for assessing apoptosis in a cell can be used, such as the Hoechst staining, Propidium Iodide (PI) or Annexin V staining), trypan blue, DNA laddering/fragmentation and TUNEL.
Flow cytometry or fluorescent microscopy assays using Omomyc specific antibodies or Omomyc labeled with suitable fluorophores.
Cell fraction autoradiography assays to identify radiolabeled Omomyc.

In a preferred embodiment, a polypeptide is considered a functionally equivalent variant of Omomyc if it shows an activity in one or more of the above assays which is at least 10%, 20%, 30%, 40%, 50%, 60%, 70%, 80%, 90% or 100% of the activity of the native Omomyc.

In a particular embodiment, the functionally equivalent variant of the polypeptide of SEQ ID NO: 1 comprises the polypeptide of SEQ ID NO: 1, wherein the residue X at position 89 of SEQ ID NO: 1 is not a cysteine. Preferably, the residue X at position 89 of SEQ ID NO: 1 is an aliphatic amino acid, or a sulfured amino acid, or a dicarboxylic amino acid or their amides, or an amino acid having two basic groups, or an aromatic amino acid, or a cyclic amino acid, or a hydroxylated amino acid. More preferably is an amino acid selected from serine, threonine and alanine, preferably selected from serine and alanine.

Suitable functionally equivalent variants of SEQ ID NO: 1 having a residue X at position 89 of SEQ ID NO: 1 which is not a cysteine are disclosed in the following table.

| SEQ | |
|---|---|
| ID | |
| NO | SEQUENCE |
| SEQ | TEENVKRRTHNVLERQRRNELKRSFFALRDQIPELENNEKAPKVV |
| ID | ILKKATAYILSVQAETQKLISEIDLLRKQNEQLKHKLEQLRNSXA |
| NO: | (wherein X is any aa different from Cys) |
| 5 | |
| SEQ | MTEENVKRRTHNVLERQRRNELKRSFFALRDQIPELENNEKAPKVV |
| ID | ILKKATAYILSVQAETQKLISEIDLLRKQNEQLKHKLEQLRNSXA |
| NO: | (wherein X is any aa different from Cys) |
| 6 | |
| SEQ | TEENVKRRTHNVLERQRRNELKRSFFALRDQIPELENNEKAPKVV |
| ID | ILKKATAYILSVQAETQKLISEIDLLRKQNEQLKHKLEQLRNSSA |
| NO: | |
| 7 | |
| SEQ | MTEENVKRRTHNVLERQRRNELKRSFFALRDQIPELENNEKAPKVV |
| ID | ILKKATAYILSVQAETQKLISEIDLLRKQNEQLKHKLEQLRNSSA |
| NO: | |
| 8 | |
| SEQ | TEENVKRRTHNVLERQRRNELKRSFFALRDQIPELENNEKAPKVV |
| ID | ILKKATAYILSVQAETQKLISEIDLLRKQNEQLKHKLEQLRNSAA |
| NO: | |
| 9 | |
| SEQ | MTEENVKRRTHNVLERQRRNELKRSFFALRDQIPELENNEKAPKVV |
| ID | ILKKATAYILSVQAETQKLISEIDLLRKQNEQLKHKLEQLRNSAA |
| NO: | |
| 10 | |

Thus, in a preferred embodiment, the functionally equivalent variant of the polypeptide of SEQ ID NO: 1 is selected from the group consisting of SEQ ID NO: 4, SEQ ID NO: 5, SEQ ID NO: 6, SEQ ID NO: 7, SEQ ID NO: 8, SEQ ID NO: 9 and SEQ ID NO: 10.

In a particular embodiment, the conjugate for use according to the invention additionally comprises a chemical moiety that facilitates cellular uptake of the polypeptide comprising SEQ ID NO: 1 or of the functionally equivalent variant of the polypeptide of SEQ ID NO: 1.

In another embodiment, the conjugate for use according to the invention does not comprise a chemical moiety that facilitates cellular uptake of the polypeptide comprising SEQ ID NO: 1 or of the functionally equivalent variant thereof.

The term “chemical moiety” refers to any chemical compound containing at least one carbon atom. Examples of chemical moieties include, but are not limited to, any peptide chain enriched in hydrophobic amino acids and hydrophobic chemical moieties.

In preferred embodiments, the conjugates according to the invention comprise at least 1, at least 2, at least 3, at least 4, at least 5, at least 6, at least 7, at least 8, at least 9, at least 10 or more chemical moieties that facilitate cellular uptake of the polypeptide or of the functionally equivalent variant of said polypeptide.

In one embodiment, the chemical moiety that facilitates cellular uptake of the polypeptide is a lipid or a fatty acid.

A fatty acid generally is a molecule comprising a carbon chain with an acidic moiety (e.g., carboxylic acid) at an end of the chain. The carbon chain of a fatty acid may be of any length, however, it is preferred that the length of the carbon chain be of at least 2, 3, 4, 5, 6, 7, 8, 9, 10, 11, 12, 13, 14, 15, 16, 17, 18, 19, 20 or more carbon atoms, and any range derivable therein. In certain embodiments, the length of the carbon chain is from 4 to 18 carbon atoms in the chain portion of the fatty acid. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated. The fatty acid may be branched, though in preferable embodiments of the present invention, it is unbranched. Specific fatty acids include, but are not limited to, linoleic acid, oleic acid, palmitic acid, linolenic acid, stearic acid, lauric acid, myristic acid, arachidic acid, palmitoleic acid, arachidonic acid.

In a preferred embodiment, the chemical moiety that facilitates cellular uptake of the polypeptide comprising SEQ ID NO: 1 or a functionally equivalent variant thereof, is a cell penetrating peptide sequence, in which case, the conjugate would comprise a fusion protein comprising the polypeptide comprising SEQ ID NO: 1 or the functionally equivalent variant thereof and the cell penetrating peptide sequence.

The term “fusion protein” relates to proteins generated by gene technology which consist of two or more functional domains derived from different proteins. A fusion protein may be obtained by conventional means, e.g., by means of gene expression of the nucleotide sequence encoding for said fusion protein in a suitable cell. It will be understood that the cell penetrating peptide refers to a cell penetrating peptide which is different from the cell penetrating peptide which forms part of the polypeptide comprising SEQ ID NO: 1 or of the functionally equivalent variant of SEQ ID NO: 1.

The term “cell penetrating peptide sequence” is used in the present specification interchangeably with “CPP”, “protein transducing domain” or “PTD”. It refers to a peptide chain of variable length that directs the transport of a protein inside a cell. The delivering process into cell commonly occurs by endocytosis but the peptide can also be internalized into cell by means of direct membrane translocation. CPPs typically have an amino acid composition that either contains a high relative abundance of positively charged amino acids such as lysine or arginine or has sequences that contain an alternating pattern of polar/charged amino acid and non-polar, hydrophobic amino acids.

Examples of CPPs which can be used in the present invention include, without limitation, the CPP found in Drosophila antennapedia protein (RQIKIWFQNRRMKWKK. SEQ ID NO:13), the CPP found in the herpesvirus simplex 1 (HSV-1) VP22 DNA-binding protein (DAATATRGRSAASRPTERPRAPARSASRPRRPVE, SEQ ID NO:14), the CPP of Bac-7 (RRIRPRPPRLPRPRPRPLPFPRPG; SEQ ID NO: 15), the CPPs of the HIV-1 TAT protein consisting of amino acids 49-57 (RKKRRQRRR, SEQ ID NO: 16), amino acids 48-60 (GRKKRRQRRRTPQ, SEQ ID NO: 17), amino acids 47-57 (YGRKKRRQRRR; SEQ ID NO: 18); the CPP of S413-PV peptide (ALWKTLLKKVLKAPKKKRKV; SEQ ID NO: 19), the CPP of penetratin (RQIKWFQNRRMKWKK; SEQ ID NO: 20), the CPP of SynB1 (RGGRLSYSRRRFSTSTGR; SEQ ID NO: 21), the CPP of SynB3 (RRLSYSRRRF; SEQ ID NO: 22), the CPP of PTD-4 (PIRRRKKLRRLK; SEQ ID NO: 23), the CPP of PTD-5 (RRQRRTSKLMKR; SEQ ID NO: 24), the CPP of the FHV Coat-(35-49) (RRRRNRTRRNRRRVR; SEQ ID NO: 25), the CPP of BMV Gag-(7-25) (KMTRAQRRAAARRNRWTAR; SEQ ID NO: 26), the CPP of HTLV-II Rex-(4-16) (TRRQRTRRARRNR; SEQ ID NO: 27), the CPP of D-Tat (GRKKRRQRRRPPQ; SEQ ID NO:28), the CPP R9-Tat (GRRRRRRRRRPPQ; SEQ ID NO: 29), the CPP of MAP (KLALKLALKLALALKLA; SEQ ID NO: 30), the CPP of SBP (MGLGLHLLVLAAALQGAWSQPKKKRKV; SEQ ID NO: 31), the CPP of FBP (GALFLGWLGAAGSTMGAWSQPKKKRKV; SEQ ID NO: 32), the CPP of MPG (ac-GALFLGFLGAAGSTMGAWSQPKKKRKV-cya; SEQ ID NO: 33), the CPP of MPG(ENLS) (ac-GALFLGFLGAAGSTMGAWSQPKSKRKV-cya; SEQ ID NO: 34), the CPP of Pep-1 (ac-KETWWETWWTEWSQPKKKRKV-cya; SEQ ID NO: 35), the CPP of Pep-2 (ac-KETWFETWFTEWSQPKKKRKV-cya; SEQ ID NO: 36), a polyarginine sequence having the structure RN (wherein N is between 4 and 17), the GRKKRRQRRR sequence (SEQ ID NO: 37), the RRRRRRLR sequence (SEQ ID NO: 38), the RRQRRTS KLMKR sequence (SEQ ID NO: 39); Transportan GWTLNSAGYLLGKINLKALAALAKKIL (SEQ ID NO: 40); KALAWEAKLAKALAKALAKHLAKALAKALKCEA (SEQ ID NO: 41); RQIKIWFQNRRMKWKK (SEQ ID NO: 42), the YGRKKRRQRRR sequence (SEQ ID NO: 43); the RKKRRQRR sequence (SEQ ID NO: 44); the YARAAARQARA sequence (SEQ ID NO: 45); the THRLPRRRRRR sequence (SEQ ID NO: 46); the GGRRARRRRRR sequence (SEQ ID NO: 47).

In a preferred embodiment, said cell-penetrating peptide is not the endogenous contained in SEQ ID NO: 1.

In a preferred embodiment, the CPP is the CPP of the HIV-1 TAT protein consisting of amino acids 49-57 (RKKRRQRRR, SEQ ID NO: 16). In another preferred embodiment the CPP is the GRKKRRQRRR sequence (SEQ ID NO: 37) or RRRRRRLR (SEQ ID NO: 38). In another embodiment the CPP is the GRKKRRQRRR sequence (SEQ ID NO: 37) or RRRRRRRR (SEQ ID NO: 65).

In some embodiments, a CPP is as a CPP as described in WO2019/018898, the content of which is incorporated herein by reference in its entirety.

In one embodiment, the cell-penetrating peptide sequence is fused at the N-terminus of the polypeptide of the invention or of the functionally equivalent variant of said polypeptide. In another embodiment, the cell-penetrating peptide is fused at the C-terminus of the polypeptide of the invention or of the functionally equivalent variant of said polypeptide.

In preferred embodiments, the conjugates or fusion proteins according to the invention comprise, in addition to the own cell penetrating peptide found in the polypeptide of SEQ ID NO: 1 or of the functionally equivalent variant of said polypeptide, at least 1, at least 2, at least 3, at least 4, at least 5, at least 6, at least 7, at least 8, at least 9, at least 10 or more additional cell penetrating peptides.

Suitable fusion proteins of the invention include the polypeptides Omomyc*TAT and Omomyc*LZArg as defined below:

| | SE | |
|---|---|---|
| | ID | |
| Name | NO: | Sequence |
| Omomyc* | 11 | MTEENVKRRTHNVLERQRRNELKRSFFALRDQIPE |
| TAT | | LENNEKAPKVVILKKATAYILSVQAETQKLISEID |
| | | LLRKQNEQLKHKLEQLRNSCAGRKKRRQRRR |
| Omomyc* | 12 | MTEENVKRRTHNVLERQRRNELKRSFFALRDQIPE |
| LZArg | | LENNEKAPKVVILKKATAYILSVQAETQKLISEID |
| | | LLRKQNEQLKHKLEQLRNSCARRRRRRLR |

Thus, in a preferred embodiment, the fusion protein is the polypeptide selected from SEQ ID NO: 11 and 12.

Suitable assays for determining whether a conjugate preserves the cell membrane translocation capacity of Omomyc include, without limitation, assays which measure the capacity of the conjugate to transduce cells in culture. This assay is based on contacting the conjugate with culture cells and detecting the presence of the conjugate in an intracellular location.

In another preferred embodiment, the conjugate of the invention additionally comprises a further nuclear localization signal.

The term “nuclear localization signal” (NLS), as used herein, refers to an amino acid sequence of about 4-20 amino acid residues in length, which serves to direct a protein to the nucleus. Typically, the nuclear localization sequence is rich in basic amino acids and exemplary sequences are well known in the art (Gorlich D. (1998) EMBO 5.17:2721-7). In some embodiments, the NLS is selected from the group consisting of the SV40 large T Antigen NLS (PKKKRKV, SEQ ID NO: 48); the Nucleoplasmin NLS (KRPAATKKAGQ AKKKK, SEQ ID NO: 49); the CBP80 NLS (RRRHSDENDGGQPHKRRK, SEQ ID NO: 50); the HIV-1 Rev protein NLS (RQARRNRRRWE, SEQ ID NO: 51); the HTLV-I Rex (MPKTRRRPRRSQRKRPPT, SEQ ID NO: 52); the hnRNP A NLS (NQSSNFGPMKGGNFGGRSSGPYGGGGQYFKPRNQGGY, SEQ ID NO: 53); the rpL23a NLS (VHSHKKKKIRTSPTFTTPKTLRLRRQPKYPRKSAPRRNKLDHY, SEQ ID NO: 54). In one embodiment of the invention, the nuclear localization signal comprises the motif K (K/R) X (K/R) (SEQ ID NO: 55).

In another preferred embodiment, the NLS may be N-terminal or C-terminal to the conjugate or the fusion protein comprising the polypeptide of SEQ ID NO: 1 or a functionally equivalent variant thereof.

The skilled person will understand that it may be desirable that the conjugate for use according to the invention further comprises one or more flexible peptides that connect the polypeptide comprising SEQ ID NO: 1 or the functionally equivalent variant thereof, the cell penetrating peptide sequence and/or the NLS. Thus, in a particular embodiment the polypeptide comprising SEQ ID NO: 1 or a functionally equivalent variant thereof is directly connected to the cell penetrating peptide sequence. In another particular embodiment, the polypeptide comprising SEQ ID NO: 1 or a functionally equivalent variant thereof is connected to the cell penetrating peptide sequence through a flexible peptide. In an embodiment the polypeptide comprising SEQ ID NO: 1 or the functionally variant thereof is directly connected to the NLS. In another embodiment the polypeptide comprising SEQ ID NO: 1 or a functionally equivalent variant thereof is connected to the NLS through flexible peptide.

In a particular embodiment the polypeptide of the conjugate for use according to the invention is directly connected to the cell penetrating peptide sequence and to the NLS.

In one embodiment, the NLS is one of the NLS which appears endogenously in the Myc sequence, such as the M1 peptide (PAAKRVKLD, SEQ ID NO: 56) or the M2 peptide (RQRRNELKRSF, SEQ ID NO: 57).

In another embodiment the additional NLS refers to an NLS which is different to the endogenous NLS found in polypeptide comprising SEQ ID NO: 1 or in the functionally equivalent variant of SEQ ID NO: 1.

In preferred embodiments, the conjugates or fusion proteins for use according to the invention comprise, in addition to the endogenous NLS found in the polypeptide of the invention or in the functionally equivalent variant thereof, at least 1, at least 2, at least 3, at least 4, at least 5, at least 6, at least 7, at least 8, at least 9, at least 10 NLS.

In another particular embodiment, the polypeptide of the conjugate for use according to the invention is connected to the cell penetrating peptide sequence through a first flexible peptide linker and to the NLS through a second flexible peptide linker.

As used herein, the term “flexible peptide”, “spacer peptide” or “linker peptide” refers to a peptide that covalently binds two proteins or moieties but which is not part of either polypeptide, allowing movement of one with respect to the other, without causing a substantial detrimental effect on the function of either the protein or the moiety. Thus, the flexible linker does not affect the tumour tracing activity of the polypeptide sequence, the cell penetrating activity of the cell penetrating peptide or the nuclear localization capacity of the NLS.

The flexible peptide comprises at least one amino acid, at least two amino acids, at least three amino acids, at least four amino acids, at least five amino acids, at least six amino acids, at least seven amino acids, at least eight amino acids, at least nine amino acids, at least 10 amino acids, at least 12 amino acids, at least 14 amino acids, at least 16 amino acids, at least 18 amino acids, at least 20 amino acids, at least 25 amino acids, at least 30 amino acids, at least 35 amino acids, at least 40 amino acids, at least 45 amino acids, at least 50 amino acids, at least 60 amino acids, at least 70 amino acids, at least 80 amino acids, at least 90 amino acids, or about 100 amino acids. In some embodiments the flexible peptide will permit the movement of one protein with respect to the other in order to increase solubility of the protein and/or to improve its activity. Suitable linker regions include a poly-glycine region, the GPRRRR sequence (SEQ ID NO: 58) of combinations of glycine, proline and alanine residues.

In an even more preferred embodiment, the nuclear localization signal is selected from the group consisting of PKKKRKV (SEQ ID NO: 48), PAAKRVKLD (SEQ ID NO: 56) and KRPAATKKAGQ AKKKK (SEQ ID NO: 49).

In a particular embodiment, the conjugates for use according to the invention comprise a tag bound to the conjugate or to the C-terminal or N-terminal domain of said polypeptide or fusion protein or variant thereof. Said tag is generally a peptide or amino acid sequence which can be used in the isolation or purification of said fusion protein. Thus, said tag is capable of binding to one or more ligands, for example, one or more ligands of an affinity matrix such as a chromatography support or bead with high affinity. An example of said tag is a histidine tag (His-tag or HT), such as a tag comprising 6 residues of histidine (His6 (SEQ ID NO: 66) or H6 (SEQ ID NO: 66)), which can bind to a column of nickel (Ni²⁺) or cobalt (Co²⁺) with high affinity. His-tag has the desirable feature that it can bind its ligands under conditions that are denaturing to most proteins and disruptive to most protein-protein interactions. Thus, it can be used to remove the bait protein tagged with H6 (SEQ ID NO: 66) following the disruption of protein-protein interactions with which the bait has participated.

Additional illustrative, non-limitative, examples of tags useful for isolating or purifying the conjugate or the polypeptide comprising SEQ ID NO: 1 or a variant thereof or a fusion protein include Arg-tag, FLAG-tag (DYKDDDDK; SEQ ID NO:59), Strep-tag (WSHPQFEK, SEQ ID NO:60), an epitope capable of being recognized by an antibody, such as c-myc-tag (recognized by an anti-c-myc antibody), HA tag (YPYDVPDYA, SEQ ID NO:61), V5 tag (GKPIPNPLLGLDST, SEQ ID NO:62), SBP-tag, S-tag, calmodulin binding peptide, cellulose binding domain, chitin binding domain, glutathione S-transferase-tag, maltose binding protein, NusA, TrxA, DsbA, Avi-tag, etc. (Terpe K., Appl. Microbiol. Biotechnol. 2003, 60:523-525), an amino acid sequence such as AHGHRP (SEQ ID NO:63) or PIHDHDHPHLVIHSGMTCXXC (SEQ ID NO:64), β-galactosidase and the like.

The tag can be used, if desired, for the isolation or purification of said fusion protein.

In a preferred embodiment, the conjugate for use according to the invention is used in the diagnosis of a lung cancer wherein the lung cancer is a primary tumor selected from small cell lung cancer (SCLC) and non small cell lung cancer (NSCLC) or is cancer metastasis.

The polypeptide comprising the sequence SEQ ID NO: 1 or a functionally equivalent variant thereof is used for the preparation of a diagnostic agent by conjugation with a detectable label.

In another aspect, the invention relates to the use of a conjugate according to the first aspect of the invention for the preparation of a diagnostic agent.

In the context of the present invention, the term “diagnostic agent” is understood to be an agent comprising a polypeptide comprising the sequence SEQ ID NO: 1 or a functionally equivalent variant thereof modified so that it may be detected after being administered to an individual.

The terms “detectable label”, “imaging agent”, “compound for imaging” and “contrast agent”, are used herein interchangeably and refer to biocompatible compounds with capacity to be detected either directly or indirectly, the use of which facilitates the differentiation of different parts of the image, by increasing the “contrast” between those different regions of the image. They refer to an atom, molecule, compound or other substance that is useful in diagnosing, detecting or visualizing cancer or other conditions associated with the uptake of the polypeptide comprising SEQ ID NO:1 or a functionally equivalent variant thereof by in vivo methods known in the art and described below.

According to the embodiments described herein, detectable labels may include, but are not limited to, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions). In addition, it should be noted that some nanoparticles, for example quantum dots and metal nanoparticles (described below) may also be suitable for use as a detection agent.

In a preferred embodiment, the detectable label is a radioactive substance, preferably a radioisotope.

Radioactive substances that may be used as detectable labels in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir. ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as detectable labels in accordance with the embodiments of the disclosure include, but are not limited to, ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu. In a more preferred embodiment, the radioactive substance or radioisotope is ⁸⁹Zr.

When the detectable label is a radioactive metal or paramagnetic ion, the label may be reacted with a reagent having a long tail with one or more chelating groups attached to the long tail for binding these ions. In that case, the chelating groups may be covalently attached to the polypeptide comprising SEQ ID NO: 1 or a functionally equivalent thereof. The long tail may be a polymer such as a polylysine, polysaccharide, or other derivatized or derivatizable chain having pendant groups to which may be bound to a chelating group for binding the ions. Examples of chelating groups that may be used according to the disclosure include, but are not limited to, ethylenediaminetetraacetic acid (EDTA), diethylenetriaminepentaacetic acid (DTPA), DOTA, NOTA, NETA, porphyrins, polyamines, crown ethers, bis-thiosemicarbazones, polyoximes, DFO (deferoxamin-maleimide) and like groups. The same chelates, when complexed with non-radioactive metals, such as manganese, iron and gadolinium are useful for MRI. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, or DFO-⁸⁹Zr to a targeting molecule for use in PET analysis. In a preferred embodiment, the chelating group is DFO. In a more preferred embodiment, the conjugate is Omomyc-DFO-⁸⁹Zr.

Enzymes that may be used as detectable labels in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

The term “contrast agents” thus encompasses agents that are used to enhance the quality of an image that may nonetheless be generated in the absence of such an agent (as is the case, for instance, in MRI), as well as agents that are prerequisites for the generation of an image (as is the case, for instance, in nuclear imaging). Suitable contrast agents include, without limitation, contrast agents for Radionuclide imaging, for computerized tomography, for Raman spectroscopy, for Magnetic resonance imaging (MRI) and for optical imaging.

Within the context of the present invention, the contrast agent may be part of the conjugate of the invention, or may be administered together with the conjugate of the invention so as to acquire nuclear medicine images that can be superimposed such us, for example, images taken with computed tomography (CT) or magnetic resonance imaging (MRI) to produce special views, a practice known as image fusion or co-registration. These views allow the information from two different exams to be correlated and interpreted on one image, leading to more precise information and accurate diagnoses. In a particular embodiment, the detection of the conjugate labelled with a radionuclide and the acquisition of a reference bodily image or section are taken simultaneously in single photon emission computed tomography/computed tomography (SPECT/CT) and positron emission tomography/computed tomography (PET/CT) units or even PET/MRI that are able to perform both imaging exams at the same time.

Contrast agents for radionuclide imaging include radiopharmaceuticals commonly labeled with positron-emitters such as ¹¹C, ¹³N, ¹⁵O, ¹⁸F, ⁸²Rb, ⁶²Cu and ⁶⁸Ga. SPECT radiopharmaceuticals are commonly labeled with positron emitters such as ⁹⁴mTc, ²⁰¹Tl and ⁶⁷Ga. Radionuclide imaging modalities (positron emission tomography, (PET); single photon emission computed tomography (SPECT)) are diagnostic cross-sectional imaging techniques that map the location and concentration of radionuclide-labeled radiotracers. PET and SPECT can be used to localize and characterize a radionuclide. In PET, radioactive conjugate that emits positrons can be monitored as the substance moves through the body. Closely related to PET is single-photon emission computed tomography, or SPECT. The major difference between the two is that instead of a positron-emitting substance, SPECT uses a radioactive tracer that emits low-energy photons.

Contrast agents for CT imaging include, for example, iodinated or brominated contrast media. Examples of these agents include iothalamate, iohexyl, diatrizoate, iopamidol, ethiodol and iopanoate. Gadolinium agents have also been reported to be of use as a CT contrast agent. For example, gadopentate agents has been used as a CT contrast agent. Computerized tomography (CT) is contemplated as an imaging modality in the context of the present invention. By taking a series of X-rays, sometimes more than a thousand, from various angles and then combining them with a computer, CT made it possible to build up a three-dimensional image of any part of the body. A computer is programmed to display two-dimensional slices from any angle and at any depth. In CT, a radiopaque contrast agent such as those described herein can assist in the identification and delineation of soft tissue masses when initial CT scans are not diagnostic.

Contrast agents for optical imaging include, for example, fluorescein, a fluorescein derivative, indocyanine green, Oregon green, a derivative of Oregon green, rhodamine green, a derivative of rhodamine green, an eosin, an erythrosin, Texas red, a derivative of Texas red, malachite green, nanogold sulfosuccinimidyl ester, cascade blue, a coumarin derivative, a naphthalene, a pyridyloxazole derivative, cascade yellow dye, dapoxyl dye and the various other fluorescent compounds disclosed herein.

In a preferred embodiment, the contrast agent is a compound that is able to be imaged by a magnetic resonance imaging apparatus. Contrast agents which can be imaged by a magnetic resonance imaging apparatus differ from those used in other imaging techniques. Their purpose is to aid in distinguishing between tissue components with identical signal characteristics and to shorten the relaxation times (which will produce a stronger signal on Tl-weighted spin-echo MR images and a less intense signal on T2-weighted images). Examples of MRI contrast agents include gadolinium chelates, manganese chelates, chromium chelates, and iron particles. In one particular embodiment, the MRI contrast agent is ¹⁹F. Both CT and MRI provide anatomical information that aid in distinguishing tissue boundaries. Compared to CT, the disadvantages of MRI include lower patient tolerance, contraindications in pacemakers and certain other implanted metallic devices, and artifacts related to multiple causes, not the least of which is motion CT, on the other hand, is fast, well tolerated, and readily available but has lower contrast resolution than MRI and requires iodinated contrast and ionizing radiation. A disadvantage of both CT and MRI is that neither imaging modality provides functional information at the cellular level. For example, neither modality provides information regarding cellular viability. Magnetic resonance imaging (MRI) is an imaging modality that is newer than CT that uses a high-strength magnet and radio-frequency signals to produce images. The most abundant molecular species in biological tissues is water. It is the quantum mechanical “spin” of the water proton nuclei that ultimately gives rise to the signal in imaging experiments. In MRI, the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. By collecting and analyzing these signals, it is possible to compute a three-dimensional image which, like a CT image, is normally displayed in two-dimensional slices.

MRI contrast agents include complexes of metals selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III). In a preferred embodiment, the compound that is able to be imaged by a magnetic resonance imaging apparatus is a gadolinium-based compound.

The term “gadolinium-based compound”, as used herein, shall mean, where used with respect to lung imaging, any gadolinium-containing substance administrable to a subject which results in an intravascular enhancement. In another embodiment, the gadolinium-containing contrast agent is selected from the group consisting of gadolinium, gadolinium pentate, and gadodiamide.

There are several forms of conjugating the detectable label to the polypeptide comprising the sequence SEQ ID NO: 1 or a functionally equivalent variant thereof. In a particular embodiment, the conjugation would be performed through maleimide-mediated methodology that is, through the binding of the maleimide-DFO (or other chelating groups) to Omomyc, or of maleimide-AF660 (or other fluorophores) to Omomyc, and the posterior maleimide reaction with the unique cysteine residue at the C-terminal of Omomyc. This coupling reaction is performed using standard procedures for maleimide labelling.

This chemical labelling step could also be performed via other chemical reagents such as NHS- (to label via free amines) or disulfide coupling agents for instance.

Detecting the presence, absence, concentration, localization or specific distribution of the conjugate for use according to the invention is accomplished by an in vivo imaging modality such as magnetic resonance imaging (MRI), positron emission tomography (PET) or microPET, computed tomography (CT), PET/CT combination imager, cooled charged coupled device (CCD), camera optical imaging, optical imaging and single photon emission computed tomography (SPECT).

In an even more preferred embodiment, the detection of the cancer cells after administration of the conjugate is performed via mPET/mCT or PET/CT imaging; preferably mPET/mCT.

The invention also provides multimodal imaging methods. Certain embodiments of the present invention pertain to methods of imaging a subject, or a site within a subject using multiple imaging modalities that involve measuring multiple signals. In certain embodiments, the multiple signals result from a single label on, or in a cell. As set forth above, any imaging modality known to those of ordinary skill in the art can be applied in these embodiments of the present imaging methods.

The imaging modalities are performed at any time during or after administration of the conjugate. For example, the imaging studies may be performed during administration of the conjugate of the invention, i.e., to aid in guiding the delivery to a specific location, or at any time thereafter.

Additional imaging modalities may be performed concurrently with the first imaging modality, or at any time following the first imaging modality. For example, additional imaging modalities may be performed about 1 sec, about 1 hour, about 1 day, or any longer period of time following completion of the first imaging modality, or at any time in between any of these stated times. In certain embodiments of the present invention, multiple imaging modalities are performed concurrently such that they begin at the same time following administration of the conjugate. One of ordinary skill in the art would be familiar with performance of the various imaging modalities contemplated by the present invention.

In some embodiments of the present methods of imaging, the same imaging device is used to perform a first imaging modality and a second imaging modality. In other embodiments, different imaging devices are used to perform the different imaging modalities. One of ordinary skill in the art would be familiar with the imaging devices that are available for performance of the imaging modalities described herein.

The instant invention provides methods for imaging cells using one or more imaging modalities. In some embodiments the conjugate for use according to the invention has more than one kind of detectable label or has multiple, equal or different, detectable labels linked to the polypeptide comprising SEQ ID NO:1 or to the functionally equivalent variant thereof. In other embodiment, the conjugate is linked with a single detectable label. In certain embodiments, the single detectable label is a multimode-detectable label.

In a preferred embodiment the detectable label is selected from the group consisting of ¹⁸F, ³²P, ³³P, ⁴⁵Ti, ⁴⁷Sc, ⁵²Fe, ⁵⁹Fe, ⁶²Cu, ⁶⁴Cu, ⁶⁷Ga, ⁶⁸Ga, ⁷⁵Sc, ⁷⁷As, ⁸⁶Y ⁸⁹Sr, ⁸⁹Zr, ⁹⁴Tc, ⁹⁴Tc, ⁹⁹mTc ⁹⁹Mo, ¹⁰⁵Pd, ¹⁰⁵Rh, ¹¹¹Ag, ¹²³I, ¹²⁴I, ¹⁴²Pr, ¹⁴³Pr, ¹⁴⁹Pm ¹⁵³Sm, ¹⁵⁴⁻¹⁵⁸¹Gd, ¹⁶¹Tb, ¹⁶⁶Dy ¹⁶⁶Ho, ¹⁶⁹Er, ¹⁷⁵Lu ¹⁸⁶Re, ¹⁸⁹Re, ¹⁹⁴Ir. ¹⁹⁸Au, ¹⁹⁹Au, ²¹¹Pb, ²¹²Bi, ²¹²Pb, ²²³Ra and ²²⁵Ac. In a more preferred embodiment, the radioactive substance or radioisotope is ⁸⁹Zr.

In an embodiment of this aspect of the invention the conjugate does not comprise a cell penetrating peptide sequence. In another embodiment the conjugate does not comprise a chemical moiety that facilitates cellular uptake of the polypeptide of SEQ ID NO: 1 or a functionally equivalent variant thereof. In another embodiment of the conjugate of the invention, the conjugate does not comprise a fluorescent label or a radioisotope, more preferably the conjugate does not comprise fluorescein-maleimide (FITC), or a radioisotope selected from the group consisting of ¹³¹I, ⁹⁰Y, ¹⁷⁷Lu, ¹⁸⁸Re, ⁶⁷Cu, ²¹¹At, ²¹³Bi, ¹²⁵I and ¹¹¹n. In another embodiment the conjugate of the invention does not comprise a cell penetrating peptide sequence, a fluorescent label or a radioisotope, more preferably does not comprise fluorescein-maleimide (FITC), or a radioisotope selected from the group consisting of ¹³¹I, ⁹⁰Y, ¹⁷⁷Lu, ¹⁸⁸Re, ⁶⁷Cu, ²¹¹At, ²¹³Bi, ¹²⁵I and ¹¹¹n.

In another embodiment, the conjugate of the invention does not comprise fluorescence groups, biotin, PEG, amino acid analogs, unnatural amino acids, phosphate groups, glycosyl groups, radioisotope labels, tags such as a histidine tag, Arg-tag, FLAG-tag, Strep-tag, an epitope capable of being recognized by an antibody, such as c-myc-tag, HA tag, V5 tag, SBP-tag, S-tag, calmodulin binding peptide, cellulose binding domain, chitin binding domain, glutathione S-transferase tag, maltose binding protein, NusA, TrxA, DsbA, Avi-tag, an amino acid sequence such as AHGHRP (SEQ ID NO: 63) or PIHDHDHPHLVIHSGMTCXXC (SEQ ID NO: 64) or β-galactosidase and the like.

The terms “diagnosis” or “detection” are used herein indistinctly and refer to the identification of the presence or characteristic of a pathological condition. The term “diagnosis”, as used herein, refers both to the process of attempting to determine and/or identify a possible disease in a subject, i.e. the diagnostic procedure, and to the opinion reached by this process, i.e. the diagnostic opinion. As such, it can also be regarded as an attempt at classification of an individual's condition into separate and distinct categories that allow medical decisions about treatment and prognosis to be made. As the person skilled in the art will understand, such a diagnosis may not be correct for 100% of the subjects to diagnose, although preferred it is. The term, however, requires that a statistically significant part of the subjects can be identified as suffering from a disease, particularly a proliferative disease in the context of the invention. The skilled in the art may determine whether a party is statistically significant using different statistical evaluation tools well known, for example, by determination of confidence intervals, the p-value determination, Student's-test, the Mann-Whitney, etc. Preferred confidence intervals are at least, 50%, at least 60%, at least 70%, at least 80%, at least 90%) or at least 95%. The p-values are preferably, 0.05, 0.025, 0.001 or lower.

In an embodiment, the method of diagnosis comprises:
a) the pulmonary administration to a subject of a conjugate comprising a polypeptide comprising the sequence SEQ ID NO: 1 or a functionally equivalent variant thereof, and a detectable label, and
b) the detection of the lung tumour in said patient by viewing the detectable label.

The expression “method of diagnosing” as referred to in accordance with the present invention means that the method may essentially consist of the aforementioned steps or may include further steps.

As used herein, the expression “in vivo diagnosis” refers to a diagnostic method applied to a human or animal body.

For the purpose of the present invention, the diagnosis is to identify the presence of lung cancer.

The terms “lung cancer” or “lung tumour” refer to the physiological condition in mammals characterized by unregulated cell growth in tissues of the lung. The term lung cancer is meant to refer to any cancer of the lung and includes non-small cell lung carcinomas and small cell lung carcinomas. In an embodiment, the lung cancer is non-small cell lung cancer (NSCLC). In another embodiment the lung cancer is small cell lung cancer (SCLC).

The term non-small cell lung cancer (NSCLC), as used herein, refers to a group of heterogeneous diseases grouped together because their prognosis and management is roughly identical and includes, according to the histologic classification of the World Health Organization/International Association for the Study of Lung Cancer (Travis W D et al. Histological typing of lung and pleural tumours. 3rd ed. Berlin: Springer-Verlag, 1999):

(i) squamous cell carcinoma (SCC), accounting for 30% to 40% of NSCLC, starts in the larger breathing tubes but grows slower meaning that the size of these tumours varies on diagnosis.

(ii) adenocarcinoma is the most common subtype of NSCLC, accounting for 50% to 60% of NSCLC, which starts near the gas-exchanging surface of the lung and which includes a subtype, the bronchioalveolar carcinoma, which may have different responses to treatment.

(iii) large cell carcinoma is a fast-growing form that grows near the surface of the lung. It is primarily a diagnosis of exclusion, and when more investigation is done, it is usually reclassified to squamous cell carcinoma or adenocarcinoma.

(iv) adenosquamous carcinoma is a type of cancer that contains two types of cells: squamous cells (thin, flat cells that line certain organs) and gland-like cells.

(v) carcinomas with pleomorphic, sarcomatoid or sarcomatous elements. This is a group of rare tumours reflecting a continuum in histologic heterogeneity as well as epithelial and mesenchymal differentiation.

(vi) carcinoid tumour is a slow-growing neuroendocrine lung tumour and begins in cells that are capable of releasing a hormone in response to a stimulus provided by the nervous system.

(vii) carcinomas of salivary gland type begin in salivary gland cells located inside the large airways of the lung.

(viii) unclassified carcinomas include cancers that do not fit into any of the aforementioned lung cancer categories.

As used herein, the term “subject” or “patient” refers to all animals classified as mammals and includes but is not limited to domestic and farm animals, primates and humans, for example, human beings, non-human primates, cows, horses, pigs, sheep, goats, dogs, cats, or rodents. Preferably, the subject is a human man or woman of any age or race.

The term “primary tumor”, as used herein, refers to a tumor that originated in the location or organ in which it is present and did not metastasize to that location from another location

In the context of the present invention, “metastasis” is understood as the propagation of a cancer from the organ where it started to a different organ. It generally occurs through the blood or lymphatic system. When the cancer cells spread and form a new tumor, the latter is called a secondary or metastatic tumor. The cancer cells forming the secondary tumor are like those of the original tumor. If a breast cancer, for example, spreads (metastasizes) to the lung, the secondary tumor is formed of malignant breast cancer cells. The disease in the lung is metastatic breast cancer and not lung cancer.

In a particular embodiment the NSCLC is selected from squamous cell carcinoma of the lung, large cell carcinoma of the lung and adenocarcinoma of the lung.

In an even more preferred embodiment, the lung cancer is adenocarcinoma.

In an even more preferred embodiment the lung adenocarcinoma is a KRAS-mutant adenocarcinoma.

KRAS refers to Kirsten rat sarcoma viral oncogene homolog (KRAS) protein. In the case of NSCLC, KRAS mutations occur predominantly (95%) at codons 12 (>80%) and 13. The most frequent codon variant, accounting for approximately 39% of KRAS-mutant NSCLCs, is the KRAS-G12C mutation. Other common mutations include KRAS-G12V (18-21%) and KRAS-G12D (17-18%) variants. Remarkably, smokers and never smokers have a different spectrum of mutations and codon variants in KRAS. Thus, transition mutations (G>A) are more frequent in never smokers, whereas transversion mutations (G>C or G>T) are more common in former or current smokers. In a preferred embodiment, the KRAS-mutant adenocarcinoma is KRAS-G12D mutant.

The term small cell lung cancer (SCLC), as used herein, refers to a proliferation of small cells with unique and strict morphological features, containing dense neurosecretory granules which give this tumor an endocrine/paraneoplastic syndrome associated. Most cases arise in the larger airways (primary and secondary bronchi). These cancers grow quickly and spread early in the course of the disease.

In another embodiment the cancer to be diagnosed by the conjugate for use according to the invention is a cancer metastasis.

The administration of the conjugate of the invention is pulmonary administration.

The term “pulmonary administration” refers to any mode of administration which delivers a pharmaceutically active substance to any surface of the lung. The modes of delivery can include, but are not limited to, a liquid suspension, as a dry powder “dust” or as an aerosol.

“Transport across a pulmonary surface” refers to any mode of passage which penetrates or permeates the interior surface of the lung. This includes passage through any lung surface, including alveolar surfaces, bronchiolar surfaces and passage between any of these surfaces. Preferably, the passage is through alveolar surfaces. Most preferably, the passage is through the type II cells on the alveolar surfaces. Passage can be either directly to the pulmonary tissues for local action or via the pulmonary tissues into the circulatory system for systemic action.

In a particular embodiment, the conjugate for use according to the invention is administered intranasally.

In an even more preferred embodiment the intranasal administration is by instillation.

In a particular embodiment, the conjugate for use according to the invention is administered by nasal instillation, nasal inhalation or oral inhalation; preferably by nasal instillation or nasal inhalation; more preferably by nasal instillation.

“Instillation” encompasses any delivery system capable of providing a effective amount of a conjugate for use according to the invention to the mammalian nares. Representative and non-limiting formats include drops, sprays, powders, aerosols, mists, catheters, tubes, syringes, applicators for creams, particulates, pellets, and the like.

The invention may be practiced with various nasal delivery vehicles and/or carriers. Such vehicles increase the half-life of the conjugate in the nares following instillation into the nares. These carriers comprise natural polymers, semi-synthetic polymers, synthetic polymers, liposomes, and semi-solid dosage forms. Natural polymers include, for example, proteins and polysaccharides. Semi-synthetic polymers are modified natural polymers such as chitosan, which is the deacetylated form of the natural polysaccharide, chitin. Synthetic polymers include, for example, dendrimers, polyphosphoesters, polyethylene glycol, poly (lactic acid), polystyrene sulfonate (PSSA), and poly (lactide coglycolide). Semi-solid dosage forms include, for example, creams, ointments, gels, and lotions. These carriers can also be used to microencapsulate the conjugate or be covalently linked to the conjugate.

In one embodiment, the conjugate for use according to the invention comprise, or is covalently or non-covalently bound to a carrier particle, which may be formulated as a powder, spray, aerosol, cream, gel, etc for application to the nares. In one embodiment, the conjugate is coated onto a carrier particle core in a dissolvable film, which may comprise a mucoadhesive. The carrier particle core may be inert, or dissolvable.

The present invention also discloses a pharmaceutical composition comprising the conjugate for use according to the invention together with a pharmaceutically acceptable carrier, which may be, for example, a powder, cream, or liquid. Pharmaceutically acceptable carriers include sterile liquids, such as water, oils, including petroleum oil, animal oil, vegetable oil, peanut oil, soybean oil, mineral oil, sesame oil, and the like. Suitable pharmaceutical carriers are described in Remington's Pharmaceutical Sciences, 18th Edition (56), incorporated by reference.

Dosage forms of compositions intended for intranasal and intrapulmonary administration are preferably a liquid, a suspension or a solid. A suspension is a liquid preparation containing solid particles dispersed in a liquid vehicle. The dosage forms are preferably metered. For examples, metered drops/sprays mean that the dispenser that includes the drops/spray delivers the drops/spray containing a metered dose (a predetermined quantity) of the conjugate for use according to the invention.

One preferred dosage form in the context of the intranasal administration route includes nasal drops. Drops are deposited mostly in the posterior portion of the nose and thus removed rapidly into the nasal pharynx. A concern with drops is often how to precisely control the drug's dose which is particularly important for the administration of the conjugate.

Another intranasal dosage form by which the conjugate for use of the invention can be administered is nasal sprays. Nasal sprays typically contain the conjugate dissolved or suspended in a solution or a mixture of excipients (e.g. preservatives, viscosity modifiers, emulsifiers, buffering agents) in a non-pressurized dispenser. Nasal sprays have several advantages including compactness of the delivery device, convenience, simplicity of use, and accuracy of delivering dosages of 25 to 200 μL. They are deposited in the anterior portion of the nose and cleared slowly into nasal pharynx by mucociliary clearance. The nasal spray as used herein can be a liquid or a suspension.

Another intranasal dosage form is a nasal aerosol. Nasal aerosols differ from nasal sprays by the method of the conjugate dispensing: in aerosols, a compound is dispensed due to an excess of pressure and releases through a valve. In sprays, a compound is dispensed due to forcing away by a micropump bucket, while the pressure in the vial is similar to atmosphere pressure. Aerosols have similar advantages as sprays.

The conjugate for use according to the invention may alternatively preferably be administered by nasal emulsions, ointments, gels, pastes or creams. These are highly viscous solutions or suspensions applied to the nasal mucosa.

Due to the limited volume of the composition that can be efficiently delivered to the nasal mucosa, liquid intranasal dosage forms usually have higher concentrations as the corresponding intravenous dosage forms. When substances become poorly soluble or are instable in liquid form, powders can be used to administer the conjugate for use of the invention. Further advantages of powders are that they do not require preservatives and have usually a higher stability as compared to liquid formulations. The main limitation on intranasal powder application is related to its irritating effect on the nasal mucosa.

One dosage form in context of intrapulmonary administration is an inhalation aerosol. Inhalation aerosols are usually packaged under pressure and contain the conjugate for use according to the invention which is released upon activation of a valve system into the respiratory tract, in particular the lungs. The released aerosol is a colloid of fine solid particles (suspension) or liquid droplets (solution) in air or another gas. Accordingly, the aerosol may be a solution or a suspension aerosol. The liquid droplets or solid particles have preferably a diameter of less than 100 μm, more preferably less than 10 μm, most preferably less than 1 μm.

Another dosage form in context of intrapulmonary administration is inhalation sprays. Inhalation sprays are typically aqueous based and do not contain any propellant. They deliver the conjugate to the lungs by oral inhalation.

Nebulized inhalation solutions and suspensions may also be used to deliver the conjugate by the intrapulmonary route. Nebulized inhalation solutions and suspensions are typically aqueous-based formulations that contain the conjugate for use according to the invention. The nebulized inhalation solutions and suspensions deliver the conjugate to the lungs by oral inhalation for systemic effects and are used with a nebulizer.

Dry powder inhalation is an alternative to aerosol inhalation. The conjugate is usually included in a capsule for manual loading or within the inhaler. Dry powders are typically delivered by an inhaler to the lungs by oral inhalation. The dry powders as used herein can be formulated neat. Neat formulations contain the drug alone or quasi-alone e.g. as spry dried powder. The dry powders as used herein can be also formulated with a carrier such as lactose.

Intrapulmonary dosage forms are preferably metered, i.e. are delivered to the lungs in a predetermined quantity.

Doses of active ingredients may be expressed either in mg of active ingredient per kg of body weight or in mg of active ingredient per square meter of body surface. The article from Reagan-Shaw S. “Dose translation from animal to human studies revisited”. FASEB J 2007, 22:659-661 provides the standard conversion factors used to convert mg/kg to mg/m2.

Dose (mg/kg)×Km=Dose (mg/m²)

This conversion is the basis for converting dose in a first animal species to dose in a second animal species (allometric dose translation). Thus, animal dose (AD) in mg/kg can be converted to human equivalent dose (HED) in mg/kg using the following formula:

$$HED ( mg / kg ) = AD ( mg / kg ) \times \frac{Animal {K}_{m}}{Human {K}_{m}}$$

wherein the Km for each species is shown in Table 1.

TABLE 1
Kₘ factor for conversion of AD to HED

| Specie | | Kₘ factor |
|---|---|---|
| Human | Adult | 37 |
| | Child | 25 |

| | |
|---|---|
| Baboon | 20 |
| Dog | 20 |
| Monkey | 12 |
| Rabbit | 12 |
| Guinea pig | 8 |
| Rat | 6 |
| Hamster | 5 |
| Mouse | 3 |

In particular, the doses mentioned herein can be adapted for any mammal according to the guidelines of the FDA for conversion of doses based on body surface area (Guidance for Industry, Estimating the Maximum Safe Starting Dose in Initial Clinical Trials for Therapeutics in Adult Healthy Volunteers, U.S. Department of Health and Human Services, Food and Drug Administration, Center for Drug Evaluation and Research (CDER), July 2005, see Table 1).

In a preferred embodiment, the dose of the conjugate for use according to the invention ranges from 0.5 to 10 mg/kg, more preferably from 1 to 5 mg/kg and more preferably from 2 to 3 mg/kg. In a preferred embodiment, the dose of the conjugate is 2.37±0.5 mg/kg, preferably 2 mg/kg, more preferably 2.37 mg/kg. In another preferred embodiment, the dose of the conjugate for use according to the invention ranges from 0.04 to 0.8 mg/Kg, more preferably from 0.08 to 0.4 mg/Kg and more preferably from 0.16 to 0.24 mg/Kg. In a preferred embodiment, the dose of the conjugate is 0.19±0.5 mg/kg, preferably 0.16 mg/kg, more preferably 0.19 mg/kg. Alternatively, the dose of the conjugate for use according to the invention ranges from 1.48 to 30 mg/m², more preferably from 3 to 14.8 mg/m² and more preferably from 5.9 to 8.8 mg/m². In a preferred embodiment, the dose of the conjugate is 7±0.5 mg/m², preferably 5.92 mg/m², more preferably 7.03 mg/m².

In a preferred embodiment, the detection of the conjugate is carried out between 30 minutes and 96 hours after the administration of the conjugate to a subject in need thereof, preferably between 30 minutes and 48 hours. In an embodiment, the detection of the conjugate is carried out at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 35 hours, at least 40 hours, at least 45 hours, at least 48 hours, at least 54 hours, at least 60 hours, at least 66 hours, at least 72 hours, at least 78 hours, at least 84 hours, at least 90 hours, at least 96 hours after the administration of the conjugate to a subject in need thereof. In a preferred embodiment, the detection of the conjugate is carried out at a time selected from 30 minutes, 1 hour, 4 hours, 24 hours and 48 hours post administration, preferably post intranasal administration.

In a preferred embodiment, after the diagnosis step, the conjugate is administered at least one more time for monitoring the progression of the lung cancer or the effect of a therapy administered to a subject suffering from lung cancer.

In the context of the present invention “monitoring the progression of the lung cancer” means to know if the size and/or extension of the lung cancer has been reduced or increased with respect to a previous measure.

In the context of the present invention “monitoring the effect of a therapy administered to a subject” means to know if a therapy administered to a subject suffering from lung cancer has reduced the size and/or extension of the lung cancer and is thus, effective; or if it has increased or not reduced the size and/or extension of the lung cancer and is, thus, ineffective. Therapies used for the treatment of lung cancer are well-known by the person skilled in the art.

In another aspect, the invention relates to the use of a polypeptide comprising the sequence SEQ ID NO: 1 or a functionally equivalent variant thereof for the preparation of a diagnostic agent.

In another aspect, the invention relates to the use of a conjugate comprising a polypeptide comprising the sequence SEQ ID NO: 1 or a functionally equivalent variant thereof and a detectable label for the preparation of a diagnostic agent.

Diagnostic and Detection Methods of the Invention

In another aspect, the invention relates to a diagnostic method for the detection of a lung tumour in a subject comprising:
i) administering by pulmonary route a conjugate comprising a polypeptide comprising the sequence SEQ ID NO:1 or a functionally equivalent variant thereof, and a detectable label,
ii) waiting a sufficient time to allow for the conjugate to enter into the proliferating lung cells,
iii) detecting the proliferating lung cells by applying an imaging technique to the subject to detect the site of accumulation of the conjugate in the subject, thereby detecting or imaging the site of proliferation, and
iv) identifying a lung tumour if a specific label is detected.

In another aspect, the invention relates to a method for diagnosing and treating a subject suspected of having lung cancer wherein said method comprises:
i) administering by pulmonary route a conjugate comprising a polypeptide comprising the sequence SEQ ID NO:1 or a functionally equivalent variant thereof, and a detectable label,
ii) waiting a sufficient time to allow for the conjugate to enter into the proliferating lung cells,
iii) detecting the proliferating lung cells by applying an imaging technique to the subject to detect the site of accumulation of the conjugate in the subject, thereby detecting or imaging the site of proliferation
iv) identifying a lung cancer if a specific labeling is detected, and
v) administering to the subject identified with lung cancer a treatment selected from surgical removal of the lung tumor and/or administration of chemotherapy and/or radiotherapy.

The term “treatment” or “treating” refers, as used herein, to the administration of a treatment to control de progression of the disease before or after the clinical signs have appeared. Control of the progression of the disease is understood as the beneficial or desired clinical results which include, but are not limited to, reduction of the symptoms, reduction of the duration of the disease, stabilization of pathological conditions (specifically avoiding additional impairment), delaying the progression of the disease, improving the pathological condition and remission (both partial and complete). The control of the progression of the disease also involves a prolongation of survival in comparison to the expected survival if the treatment was not applied.

The expression “site of accumulation of the conjugate”, as used herein, refers to the site wherein Omomyc or a functionally equivalent variant thereof is located after pulmonary administration. The visualization of the site of accumulation is achieved by means of the label forming part of the conjugate.

The expression “site of proliferation”, as used herein, refers to the site wherein the cancer is located, i.e., to the site wherein proliferating cells are located.

The expression “surgical removal”, as used herein, refers to the surgical removal of the lung cancer tissue and some surrounding tissue. The lung can be completely or partially removed. Two common used approaches to remove portions of the lung are without limitation thoracotomy and minimally invasive surgery. Exemplary lung surgical removal in patients suffering from cancer are without limitation lobectomy, segmentectomy, wedge resection or pneumonectomy.

The term “chemotherapy” refers to the use of drugs to destroy cancer cells. The drugs are generally administered through oral or intravenous route. Sometimes, chemotherapy is used together with radiation treatment. Chemotherapy refers to a treatment with an antineoplastic drug used to treat cancer or the combination of more than one of these drugs into a cytotoxic standardized treatment regimen. In the context of the present invention, the term “chemotherapy” comprises any antineoplastic agent including small sized organic molecules, peptides, oligonucleotides and such like used to treat lung cancer as well as related processes such as angiogenesis or metastasis. Suitable chemotherapy agents include but are not limited to alkylating agents [e.g., Cisplatin, Carboplatin, Oxaliplatin, BBR3464, Chlorambucil, Chlormethine, Cyclophosphamides, Ifosfamide, Melphalan, Carmustine, Fotemustine, Lomustine, Streptozocin, Busulfan, Dacarbazine, Mechlorethamine, Procarbazine, Temozolomide, ThioTPA, Uramustine, etc.]; antimetabolites [e.g., purine (azathioprine, mercaptopurine), pyrimidine (Capecitabine, Cytarabine, Fluorouracil, Gemcitabine), folic acid (Methotrexate, Pemetrexed, Raltitrexed), etc.]; vinca alkaloids [e.g., Vincristine, Vinblastine, Vinorelbine, Vindesine, etc.]; taxanes [e.g., paclitaxel, docetaxel, BMS-247550, etc.]; anthracyclines [e.g., Daunorubicin, Doxorubicin, Epirubicin, Idarubicin, Mitoxantrone, Valrubicin, Bleomycin, Hydroxyurea, Mitomycin, etc.]; topoisomerase inhibitors [e.g., Topotecan, Irinotecan Etoposide, Teniposide, etc.]; monoclonal antibodies [e.g., Alemtuzumab, Bevacizumab, Cetuximab, Gemtuzumab, Panitumumab, Rituximab, Trastuzumab, etc.]); photosensitizers [e.g., Aminolevulinic acid, Methyl aminolevulinate, Porfimer sodium, Verteporfin, etc.]; tyrosine kinase inhibitors [e.g., imatinib]; epidermal growth factor receptor inhibitors [e.g., erlotinib, gefitinib, etc.]; FPTase inhibitors [e.g., FTIs (RI 15777, SCH66336, L-778, 123), etc.]; KDR inhibitors [e.g., SU6668, PTK787, etc.]; proteasome inhibitors [e.g., PS341, etc.]; TS/DNA synthesis inhibitors [e.g., ZD9331, Raltitrexed (ZD 1694, Tomudex), ZD9331, 5-FU, etc.]; S-adenosyl-methionine decarboxylase inhibitors [e.g., SAM468A, etc.]; DNA methylating agents [e.g., TMZ, etc.]; DNA binding agents [e.g., PZA, etc.]; agents which bind and inactivate O-6-alkylguanine AGT [e.g., BG]; c-ra/-I antisense oligo-deoxynucleotides [e.g., ISIS-5132 (CGP-69846A)]; tumor immunotherapy; steroidal and/or nonsteroidal antiinflammatory agents [e.g., corticosteroids, COX-2 inhibitors]; immune checkpoint inhibitors (PD-L1 inhibitors such as atezolizumab or durvalumab; or PD-1 inhibitors such as nivolumab, pembrolizumab; or CTLA-4 inhibitors such as ipilimumab); or other agents such as Alitretinoin, Altretamine, Amsacrine, Anagrelide, Arsenic trioxide, Asparaginase, Bexarotene, Bortezomib, Celecoxib, Dasatinib, Denileukin Diftitox, Estramustine, Hydroxycarbamide, Imatinib, Pentostatin, Masoprocol, Mitotane, Pegaspargase, and Tretinoin.

Platinum-based compounds are specially suitable for treating lung cancer including, without limitation, carboplatin, cisplatin [cis-diamminedichloroplatinum, (CDDP)], oxaliplatin, iproplatin, nedaplatin, triplatin tetranitrate, tetraplatin, satraplatin (JM216), JM118 [cis ammine dichloro (II)], JM149 [cis ammine dichloro (cyclohexylamine) trans dihydroxo platinum (IV)], JM335 [trans ammine dichloro dihydroxo platinum (IV)], transplatin, ZD0473, cis, trans, cis-Pt(NH3)(C6H11 NH2)(OOCC3H7)2Cl, malanate-1,2-diaminociclohexanoplatin(II), 5-sulphosalycilate-trans-(1,2-diaminociclohexane)platin (II) (SSP), poly-[(trans-1,2-diaminocyclohexane)platin]-carboxyamilose (POLY-PLAT) and 4-hydroxy-sulphonylphenylacetate (trans-1,2-diaminocyclohexane) platinum (II) (SAP) and the like. In a particular embodiment, the platinum-based compound is selected from carboplatin, cisplatin and oxaliplatin; preferably is cisplatin.

Suitable combinations for the treatment of lung cancer, particularly NSCLC, can be, without limitation, a platinum-based compound and a mitotic inhibitor (for example, cisplatin-paclitaxel, cisplatin-docetaxel (CI-TA regimen), carboplatin-paclitaxel, carboplatin-docetaxel, oxaliplatin-paclitaxel, cisplatin-vinorelbine, carboplatin-vinorelbine, cisplatin-vindesine, oxaliplatin-vinorelbine); a platinum-based compound and an antimetabolite (for example, cisplatin-gemcitabine, carboplatin-gemcitabine, oxaliplatin-gemcitabine); a platinum-based compound, a mitotic inhibitor and an anti-VEGF drug (for example, cisplatin-docetaxel-bevacizumab); a platinum-based compound, an antimetabolite and an anti-VEGF drug (for example, cisplatin-gemcitabine-bevacizumab). Other suitable combinations are cisplatin-etoposide, carboplatin-etoposide, cisplatin-teniposide, cisplatin-vindesine, cisplatin-tirapazamine, ZD0473-vinorelbine, ZD0473-paclitaxel, ZD0473-gemcitabine, cisplatin-etoposide-mitomycin C, cisplatin-paclitaxel-gemcitabine, cisplatin-doxorubicin-5-fluorouracil (AFP), cisplatin-cyclophosphamide-bleomycin (CBP), cisplatin-vindesine-mitomycin C (MVP), cyclophosphamide-doxorubicin-cisplatin (CISCA), cisplatin-adriamycin (CA), cisplatin-fluorouracil (CF), cisplatin-gemcitabine-vinorelbine (CGV regimen), and paclitaxel followed by cisplatin-gemcitabine-vinorelbine (T-CGV regimen).

In a preferred embodiment, the chemotherapy agent is Omomyc, particularly is a polypeptide comprising the sequence SEQ ID NO: 1 or a functionally equivalent variant thereof, preferably disclosed in the context of the first aspect of the invention or disclosed in patent applications WO 2014/180889 A1 and WO 2018/011433 A1.

The term “radiotherapy” refers to therapy that delivers radiation that can destroy rapidly dividing cells or to palliate symptoms. Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation).

In another aspect the invention relates to a method for detecting or imaging lung cancer cells in a subject comprising:
i) administering intranasally a conjugate comprising a polypeptide comprising the sequence SEQ ID NO:1 or a functionally equivalent variant thereof, and a detectable label,
ii) waiting a sufficient time to allow for the conjugate to enter into the proliferating lung cells.
iii) detecting the proliferating lung cells by applying an imaging technique to the subject to detect the site of accumulation of the conjugate in the subject, thereby detecting or imaging the site of proliferation.

“Detecting” refers to determining the presence, absence, or amount of an analyte in a sample, and can include quantifying the amount of the analyte in a sample or per cell in a sample.

In another aspect, the invention relates to a method of imaging lung cancer using a conjugate comprising:
i) a polypeptide comprising the sequence SEQ ID NO: 1 or a functionally equivalent variant thereof, and
ii) a detectable label,
   wherein said conjugate is administered by pulmonary route.

All the embodiments disclosed in the context of the diagnostic uses of the invention are also applicable to the diagnostic and detection methods of the invention.

Monitoring Uses of the Conjugate of the Invention

In another aspect, the invention relates to a method for monitoring the progression of lung cancer in a subject, the method comprising:
i) administering pulmonary a conjugate comprising a polypeptide comprising the sequence SEQ ID NO: 1 or a functionally equivalent variant thereof, and a detectable label;
ii) waiting a sufficient time to allow for the conjugate to enter into the proliferating lung cells; and
iii) detecting the proliferating lung cells by applying an imaging technique to the subject to detect the site of accumulation of the conjugate in the subject, thereby detecting or imaging the site of proliferation
iv) comparing the site of accumulation obtained in iii) with the site of accumulation obtained in a previous measure.
wherein a significant decrease or lack of change in the site of accumulation in comparison with the previous measure is indicative that the lung cancer is not progressing or
wherein a significant increase in the site of accumulation in comparison with the previous measure is indicative that the lung cancer is progressing.

The expression “monitoring the progression of lung cancer”, as used herein, refers to the determination of the progression of the disease or the prediction of the probable course and outcome of a clinical condition or disease in a patient diagnosed with lung cancer (e.g., worsening, partial recovery or complete recovery). A prognosis of a patient is usually made by evaluating factors or symptoms of a disease that are indicative of a favorable or unfavorable course or outcome of the disease. It is understood that the term prognosis does not necessarily refer to the ability to predict the course or outcome of a condition with 100% accuracy. Instead, the skilled artisan will understand that the term “prognosis” refers to an increased probability that a certain course or outcome will occur. In the context of the invention, monitoring the progression of lung cancer refers to use imaging to evaluate whether a lung tumour is shrinking or growing.

According to this aspect, the site of accumulation of the conjugate of the invention at a first period of time (first subject sample) and the site of accumulation at a second period of time (second subject sample) are compared allowing the progression of lung cancer to be monitorized. The second subject sample can be taken from the same subject having lung cancer from which the first measure is derived, at a second period of time, i.e., at any time after the first period of time, e.g., one day, one week, one month, two months, three months, 1 year, 2 years, or more after the first subject sample.

In a particular embodiment, the first subject sample is taken prior to the subject receiving treatment, e.g., chemotherapy, radiation therapy, or surgery, and the second subject sample is taken after treatment. In another particular embodiment, the first subject sample is taken after the subject has started/received treatment, e.g., chemotherapy, radiation therapy, or surgery, and the second subject sample is taken later, at different time periods during a course of treatment. Consequently, if the lung cancer progresses or has bad prognosis, a further therapy should be designed to treat said disease in said subject.

Methods to obtain an image enabling localization of the site of accumulation of the conjugate and methods to quantify the site of accumulation or to calculate dimensions or mass of the tumour are known by the person skilled in the art. The amount of the conjugate accumulated in the tumor site after pulmonary administration of the conjugate will be indicative of tumor size. The comparison of that accumulation over time in consecutive scans performed in the same patient correlates with disease progression.

Once the site of accumulation in the subject samples, at different periods of time (first and second subject samples) has been measured, it is necessary to identify if there is a significant increase, decrease or lack of change in the site of accumulation in the second subject sample in comparison with the site of accumulation in the first subject sample. This is done by comparing the first site of accumulation and the second site of accumulation.

In the context of the present invention, a decrease in the site of accumulation in the second subject sample under study is considered a significant decrease with respect to the site of accumulation in the first subject sample when the site of accumulation in the second subject sample decreases with respect to the first subject sample by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% (i.e., absent).

In the context of the present invention, an increase in the site of accumulation in the second subject sample under study is considered a significant increase with respect to the site of accumulation in the first subject sample when the site of accumulation in the second subject sample increases with respect to the first subject sample by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150% or more.

Similarly, a lack of change in the site of accumulation in the second subject sample under study with respect to the first subject sample is considered when the site of accumulation in the second subject sample indicates that the site of accumulation is substantially constant between the two measurements. By way of example, constant accumulation indicates that the first measurement is not more than 105%, not more than 104%, not more than 103%, not more than 102%, not more than 101%, not less than 99%, not less than 98%, not less than 97%, not less than 97%, not less than 96% or not less than 95%.

Thus, a significant increase in the second subject sample with respect to the first subject sample is indicative that lung cancer is in progression (i.e., it has a bad prognosis); thus the therapy administered to the subject under study should be changed and a new therapy should be designed to treat lung cancer in said subject. On the contrary, if no significant increase in the second subject sample with respect to the first subject sample is achieved, or even if a significant decrease in the second subject sample with respect to the first subject sample is achieved, then lung cancer is not in progression (i.e., it does not have a bad prognosis).

In another aspect, the invention relates to a method for monitoring the response to a therapy in a subject suffering from lung cancer, the method comprising:
i) administering pulmonary a conjugate comprising a polypeptide comprising the sequence SEQ ID NO: 1 or a functionally equivalent variant thereof, and a detectable label;
ii) waiting a sufficient time to allow for the conjugate to enter into the proliferating lung cells; and
iii) detecting the proliferating lung cells by applying an imaging technique to the subject to detect the site of accumulation of the conjugate in the subject, thereby detecting or imaging the site of proliferation
iv) comparing the site of accumulation obtained in iii) with the site of accumulation obtained in a previous measure prior to administration of the therapy
wherein a significant decrease or lack of change in the site of accumulation in comparison with the previous measure is indicative that the therapy administered to the subject is efficacious or
wherein a significant increase in the site of accumulation in comparison with the previous measure is indicative that the therapy administered to the subject is inefficacious.

The expression “monitoring the response to a therapy”, as used in the present invention, relates to the possibility of determining the response of a subject suffering from lung cancer to the therapy administered to said subject. In lung cancer therapy, a variety of treatments can be used in an attempt to eliminate or contain the cancer. Treatment for lung cancer may involve active surveillance, surgical removal, chemotherapy, radiotherapy or some combination. Which option is best depends on many factors. Because all treatments can have significant side effects, treatment discussions often focus on balancing the goals of therapy with the risks of lifestyle alterations.

Therefore, in this aspect, the first subject sample is taken prior to the administration of the therapy, and the second subject sample is taken after the subject has started/received treatment, e.g., chemotherapy, surgery or radiotherapy. This method allows for the evaluation of a particular treatment for a selected subject previously diagnosed with lung cancer. Consequently, if the therapy is not efficacious for treating lung cancer in said subject (the patient is not improving), then said therapy should be changed and a new therapy should be designed to treat lung cancer in said subject. The course of the new treatment can be easily followed according to these methods. On the opposite, if the patient is improving may be maintained on the current therapy.

The expressions “significant decrease”, “significant increase” or “lack of change” in the site of accumulation have been defined previously.

In the context of the present invention, the therapy administered to the subject is efficacious when it leads to reduced accumulation of the conjugate of the invention in the lung of a subject suffering from lung cancer and being treated with said therapy, that is, when after the administration there is a reduction or elimination of all measurable lesions.

Ideally, all uni- or bidimensionally measurable lesions should be measured at each assessment. In a preferred embodiment, the patient can show a complete response or a partial response.

In an embodiment, complete response means a complete disappearance of all clinically detectable malignant disease, determined by two separate assessments.

In another embodiment, partial response means a reduction from baseline in the sum of the products of the longest perpendicular diameters of all measurable disease without progression of evaluable disease and without evidence of any new lesions as determined by two consecutive assessments. In an embodiment partial response can be at least a 5% reduction. More preferably, the partial response means a 50% or greater reduction.

In the context of the present invention, the therapy administered to the subject is inefficacious when it does not help to reduce the accumulation of the conjugate of the invention in the lung of a subject suffering from lung cancer and being treated with said therapy, that is, when after the administration there is an increase of all measurable lesions.

In another aspect, the invention relates to the use of a conjugate comprising a polypeptide comprising sequence SEQ ID NO: 1 or a functionally equivalent variant thereof, and a detectable label, in a method for monitoring the progression of lung cancer or for monitoring the response to a therapy in a subject suffering from lung cancer. In a particular embodiment, the conjugate is administered pulmonary.

In another aspect, the present invention provides a method for treating a subject suffering from lung cancer, comprising administering a treatment selected from surgical removal of the lung tumor and/or administration of chemotherapy and/or radiotherapy, wherein the subject is identified by a diagnosis, detecting, or imaging method as described herein.

In another aspect, the present invention provides a method for treating a subject suffering from lung cancer, comprising administering a treatment selected from surgical removal of the lung tumor and/or administration of chemotherapy and/or radiotherapy, wherein the progression of lung cancer in the subject is assessed by a monitoring method as described herein.

### KITS OF THE INVENTION

In another aspect the invention relates to a kit for the diagnosis of lung cancer comprising:
i) a conjugate comprising a polypeptide comprising the sequence SEQ ID NO:1 or a functionally equivalent variant thereof, and a detectable label,
ii) a device for the nasal instillation or nasal inhalation of the conjugate of item i); and
iii) means for packaging items i) and ii).

In another aspect, the invention relates to the use of a kit according to the invention for diagnosing lung cancer or for monitoring the progression of lung cancer or for monitoring the effect of a therapy.

Also encompassed within the invention are kits comprising a composition containing the conjugate for use according to the invention, in connection with an appropriate delivery device or applicator for the composition, for example: catheters, tubes, sprayers, syringes, atomizers, or other applicator for creams, particulates, pellets, powders, liquids, gels and the like.

Devices for intranasal delivery in the context of the present invention include spray pump systems, pipettes for delivering drops, metered-dose spray pumps, nasal pressurized metered-dose inhalers, powder spray systems, breath-actuated powder inhalers and nasal powder insufflators. The intranasal delivery device may be filled with a single dose amount or a multi-dose amount of the intranasal formulation.

Using the intrapulmonary route the conjugate may be administered with a metered dose inhaler. A metered-dose inhaler (MDI) provides a fine mist of conjugate, generally with an aerodynamic particle size of less than 5 μm.

Dry powder inhalers can be alternatively used to deliver the conjugate intrapulmonary. Dry powder inhalers present powders as single-dose or multidose powders.

Another device for intrapulmonary delivery is a nebulizer including ultrasonic and air jet nebulizers. In ultrasonic nebulizers, ultrasound waves are formed in an ultrasonic nebulizer chamber by a ceramic piezoelectric crystal that vibrates when electrically excited. This generates an aerosol cloud at the solution surface. The aerosol produced by an air jet nebulizer is generated when compressed air is forced through an orifice. A liquid may be withdrawn from a perpendicular nozzle (the Bernoulli Effect) to mix with the air jet which is atomized using baffles to facilitate the formation of the aerosol cloud.

All the embodiments disclosed in the context of the previous aspects of the invention are also applicable to the kits of the invention.

Conjugate of the Invention

In another aspect, the invention relates to a conjugate comprising:
i) a polypeptide comprising the sequence SEQ ID NO: 1 or a functionally equivalent variant thereof, and
ii) a detectable label, preferably selected from the group consisting of a contrast agent or an imaging agent.

In a preferred embodiment of this aspect of the invention the conjugate does not comprise a cell penetrating peptide sequence. In another preferred embodiment the conjugate does not comprise a chemical moiety that facilitates cellular uptake of the polypeptide of SEQ ID NO: 1 or a functionally equivalent variant thereof. In another preferred embodiment of the conjugate of the invention, the conjugate does not comprise a fluorescent label or a radioisotope, more preferably the conjugate does not comprise fluorescein-maleimide (FITC), or a radioisotope selected from the group consisting of ¹³¹I, ⁹⁰Y, ¹⁷⁷Lu, ¹⁸⁸Re, ⁶⁷Cu, ²¹¹At, ²¹³Bi, ¹²⁵I and ¹¹¹In. In a preferred embodiment the conjugate of the invention does not comprise a cell penetrating peptide sequence, a fluorescent label or a radioisotope, more preferably does not comprise fluorescein-maleimide (FITC), or a radioisotope selected from the group consisting of ¹³¹I, ⁹⁰Y, ¹⁷⁷Lu, ¹⁸⁸Re, ⁶⁷Cu, ²¹¹At, ²¹³Bi, ¹²⁵I and ¹¹¹In.

In another embodiment, the conjugate of the invention does not comprise fluorescence groups, biotin, PEG, amino acid analogs, unnatural amino acids, phosphate groups, glycosyl groups, radioisotope labels, tags such as a histidine tag, Arg-tag, FLAG-tag, Strep-tag, an epitope capable of being recognized by an antibody, such as c-myc-tag, HA tag, V5 tag, SBP-tag, S-tag, calmodulin binding peptide, cellulose binding domain, chitin binding domain, glutathione S-transferase tag, maltose binding protein, NusA, TrxA, DsbA, Avi-tag, an amino acid sequence such as AHGHRP (SEQ ID NO: 63) or PIHDHDHPHLVIHSGMTCXXC (SEQ ID NO: 64) or β-galactosidase and the like.

In a preferred embodiment the contrast agent or imaging agent is selected from the group consisting of ¹⁸F, ³²P, ³³P, ⁴⁵Ti, ⁴⁷Sc, ⁵²Fe, ⁵⁹Fe, ⁶²Cu, ⁶⁴Cu, ⁶⁷Ga, ⁶⁸Ga, ⁷⁵Sc, ⁷⁷As, ⁸⁶Y ⁸⁹Sr ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹²³I, ¹²⁴I, ¹⁴²Pr, ¹⁴³Pr, ¹⁴⁹Pm ¹⁵³Sm, ¹⁵⁴⁻¹⁵⁸¹Gd, ¹⁶¹Tb, ¹⁶⁶Dy ¹⁶⁶Ho, ¹⁶⁹Er, ¹⁷⁵Lu ¹⁸⁶Re, ¹⁸⁹Re, ¹⁹⁴Ir. ¹⁹⁸Au, ¹⁹⁹Au, 211 Pb, ²¹²Bi, 15 ²¹²Pb, ²²³Ra and ²²⁵Ac. In a more preferred embodiment, the radioactive substance or radioisotope is ⁸⁹Zr.

All the embodiments disclosed in the context of the previous aspects of the invention are also applicable to the conjugates of the invention.

All terms as used herein, unless otherwise stated, shall be understood in their ordinary meaning as known in the art. Other more specific definitions for certain terms as used in the present application are as set forth below and are intended to apply uniformly throughout the description and claims unless an otherwise expressly set out definition provides a broader definition. Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. Furthermore, the present invention covers all possible combinations of particular and particular embodiments described herein.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). The term “about” as used in connection with a numerical value throughout the specification and the claims denotes an interval of accuracy, familiar and acceptable to a person skilled in the art. In general, such interval of accuracy is ±15%. Unless defined otherwise, all technical and scientific terms used herein have the same meaning as commonly understood by one of ordinary skill in the art to which this disclosure is related. Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

The invention will be described by way of the following examples which are to be considered as merely illustrative and not limitative of the scope of the invention.

### EXAMPLES

Methodology

Production, Purification and Labelling of Omomyc

The Omomyc peptide sequence (SEQ ID NO: 1) was reverse transcribed, codon optimized for expression in E. coli adding a Methionine in the N-terminal end, cloned in a pET3a expression vector (Novagen) and purified from BL21 (DE3) Arabinose-Inducible (Invitrogen®) bacterial strain using protocols adapted from the Max° purification protocol described in J.-F. Naud et al. 2003. J Mol Biol, 326:1577-1595; F.-O. and Mcduff et al. 2009. J Mol Recognit, 22:261-269). The purified construct obtained was the polypeptide of SEQ ID NO: 4. Identity of each purified construct was confirmed by mass spectrometry and by western blot analysis. Maleimide conjugation with AlexaFluor®660- (Invitrogen) or deferoxamin- (Macrocyclics) moieties to the unique C-terminal cysteine residue of Omomyc was performed according to the manufacturers' indications. The covalently modified peptides were purified from the free labelling agent by cationic exchange followed by size exclusion chromatography, and the complete labelling and purity was confirmed by mass spectrometry analysis, SDS-PAGE and UV spectroscopy. For the in vivo administrations, an additional purification step was carried out in order to remove endotoxins using the ToxinEraser™ Endotoxin Removal Kit (Genscript). Endotoxin concentrations were quantified using the Pierce® LAL Chromogenic Endotoxin Quantification Kit (Thermo Scientific). Buffer exchange was carried out in Amicon Ultra-15 (MerckMillipore) with a 3 kDa exclusion limit.

For radiolabeling of Omomyc-DFO, ⁸⁹Zr (T_{1/2}=78.4 h, p+=22.6%; ˜2.7 GBq/mL supplied in 1 M oxalic acid) was obtained from BV Cyclotron VU (Amsterdam, The Netherlands). The required volume of ⁸⁹Zr-oxalic acid solution corresponding to 74 MBq was adjusted to a total volume of 200 μL using 1 M oxalic acid; 90 μL of 2 M Na₂CO₃ were added and incubated for 3 min at room temperature. One mL of 0.5 M HEPES and 710 μL of Omomyc-DFO (2.17 mg/mL) were added and incubated at room temperature for 60 min on a rotating shaker; pH was checked to be at 7.0-7.5. The reaction mixture was loaded on a previously equilibrated PD-10 column and eluted with phosphate-buffered saline (PBS) into fractions of 500 μL. The collected fractions were measured in a dose calibrator (IBC, Veenstra Instruments). Quality control was performed by instant thin-layer chromatography (ITLC) on ITLC strips (model 150-771, Biodex) using 0.02 M citrate buffer (pH 5.0):acetonitrile (9:1) as eluent. Stability of Omomyc-DFO-⁸⁹Zr was investigated by incubation in human serum (HS), PBS and DTPA (50 mM) for 24 h at 37° C. The radiochemical purity was determined by ITLC as explained above. The radiochemical yield, purity, and specific activity of the Omomyc-DFO-⁸⁹Zr used in this study were >98%, >99% and 34 MBq/mg respectively, assuming virtually complete recovery of the Omomyc-DFO-⁸⁹Zr conjugates after size exclusion chromatography; therefore, the compound was used with no further purification. Stability studies in HS and PBS at 37° C. showed that more than 99% of the radiotracer remained intact after 24 h, whereas in DTPA, more than 96% of the radiotracer remained intact after 24 h at 37° C., supporting that Omomyc-DFO-⁸⁹Zr constitutes a suitable probe for in vivo studies.

Pharmacokinetic and Biodistribution Study

For the pharmacokinetic and biodistribution studies by microPET/CT, female 8-week old FVB/NRj mice were purchased from JANVIER LABS. Experiments were carried out in compliance with National Guidelines for Animal Protection and the approval of the regional animal care committee and Animal Ethical Committee of CIEMAT. Mice were housed in an animal facility at the CIEMAT.

For the i.n. administration studies, one group of 5 mice receiving isoflurane anesthesia was removed from the induction chamber and i.n. administration was performed immediately by pipetting 30 μl of Omomyc-DFO-⁸⁹Zr (2.9±0.4 MBq, 2.37±0.5 mg Omomyc-DFO/kg body weight) onto the outer edge of nares. Mice were euthanized 48 h after injection by cervical dislocation under anesthesia with isoflurane in O₂ and blood was immediately collected by cardiac puncture. For biodistribution studies mice were imaged at indicated time points by PET/mCT imaging as described below For biodistribution studies organ tissues were excised, wet-weighed and counted for radioactivity with a gamma-counter (2470 Wizard², PerkinElmer), along with a standard sample of the injected dose. Grubbs test was applied to detect the presence of one outlier animal in the global dataset and the data from this outlier were discarded.

For the i.v. administration studies, one group of 5 Balb/c-nude mice bearing established subcutaneous xenografts of H1975 cells (average size 430 mm³) was administered with Omomyc-DFO-⁸⁹Zr (3.2±0.1 MBq, 2.62±0.3 mg Omomyc-DFO/kg body weight) in the tail vein. Mice were euthanized at 72 h after injection by cervical dislocation under anesthesia with isoflurane in O₂ and blood was immediately collected by cardiac puncture. For biodistribution studies mice were imaged at indicated time points by PET/mCT imaging as described below. At endpoint, organ tissues were excised, wet-weighed and counted for radioactivity with a gamma-counter (2470 Wizard², PerkinElmer), along with a standard sample of the injected dose, at 48 h post-administration.

For the biodistribution and lung adenocarcinoma treatment studies, a minimum of 5 mice per time point and condition were randomized and treatment started 16 weeks after adeno-CRE infection. Animals were anesthetized by inhaled isoflurane (AbbVie Farmaceutica S.L.U.) and intranasally administered a single dose of 1.4 mg/kg of OmomycCPP-AF660 or the vehicle (10 mM sodium acetate pH 6.5) in 30 μL total volume and euthanized at the indicated time points. Tissues were harvested and the fluorescence immediately visualized by IVIS® Spectrum imaging using 663 nm and 690 nm wavelengths for excitation and emission respectively. Measurements of fluorescent signals were acquired and analyzed using Living Image® 4.3.1 software (PerkinElmer).

### MicroPET/CT Imaging

After i.n. instillation, mice were scanned immediately with a small-animal Argus PET-CT scanner (SEDECAL, Madrid, Spain). The PET studies (energy window 400-700 KeV and 20 min static acquisition) and CT (voltage 45 kV, current 150 μA, 8 shots, 360 projections and standard resolution) were performed at various time points post-injection (30 min, 4, 24 and 48 hours) in mice anesthetized by inhalation of 2-2.5% isoflurane. The PET images were reconstructed using a 2D-OSEM (Ordered Subset Expectation Maximization) algorithm (16 subsets and two iterations), with random and scatter correction. A calibration factor predetermined by scanning a cylindrical phantom containing a known activity of ⁸⁹Zr was used to convert counts per pixel/sec to kBq/cm³. Manually drawn regions of interest (ROIs) in PET images (intranasal delivery, oral cavity and oropharyngeal region, esophagus and gut) or ROIs selected from PET images using CT anatomical guidelines (for lung, liver and kidneys) were used to determine the mean radiotracer accumulation in units of % ID/g tissue (decay corrected to the time of injection) by dividing the obtained average tracer concentration (kBq/cm³) in the region by the total ID (kBq). Percentage-injected dose in a ROI was calculated by multiplying the obtained average tracer concentration (kBq/cm³) by the ROI volume (cm³). Separate image calibration factors were also determined for lung, kidneys and liver by comparing the final scans (48 h) with direct assays of organs performed after the animals were euthanized. These calibration factors were used to normalize the % ID/g obtained from PET imaging to activity concentrations at different points in time after injection. Images were analyzed using the image analysis software ITK-SNAP (www.itksnap.org).

Immunohistochemistry

Mice were euthanized by cervical dislocation. Lungs were excised and perfused through the trachea with PBS followed by 3.7% PFA, fixed overnight, transferred to 70% ethanol and embedded in paraffin. Sections were cut at 4 micrometers thick and stained with H&E for pathological examination. For anti-Omomyc immunohistochemistry, antigen retrieval was performed by heating 20 min at 400 W in a microwave in 0.01 M citrate buffer pH 6.0. After blocking 45 minutes in 3% BSA and washing in PBS, slides were incubated overnight at 4° C. with the primary rabbit polyclonal anti-Omomyc antibody (affinity purified and selected against recognition of MYC epitope) at a 0.02 mg/mL final concentration. After a PBS wash, slides were incubated with goat anti-rabbit IgG (H+L)-AlexaFluor®488 conjugate (Thermo Fisher Scientific A-11008) diluted 1/200 and stained with DAPI (Life Technologies D1306) diluted 1/10000, washed once with water and mounted with Fluorescence Mounting Medium (Dako S3023).

Mice

KRas^{LSL-G12D/+} mice were genotyped by Transnetyx and generation of lung tumors in both males and females was performed as described previously (E. L. Jackson. 2001. Genes & Development, 15:3243-3248). Animals were maintained in a mixed C57BL/6J×FVBN background. A minimum of 5 mice per time point and condition were randomized and treatment started 16 weeks after Adeno-Cre infection (two biological replicates of the experiment were performed). Animals were anesthetized by inhaled isoflurane (AbbVie Farmaceutica S.L.U.) and were i.n. administered with either Omomyc or the vehicle (10 mM sodium acetate pH 6.5) in 30 μL total volume.

Statistical Analysis

All analyses and graphs were performed using GraphPad Prism 5 software. Normal distribution of the data was assessed for each group using D'Agostino-Pearson test. Differences in samples' average values were analyzed using Student t-test or ANOVA (parametric) for normally distributed data or Mann-Whitney or Kruskal-Wallis test when otherwise. F test was used to calculate the difference in the variances of the groups.

### Example 1: The Omomyc Mini-Protein Localizes Mainly in Lung Tumors Following Direct Pulmonary Administration in a Mouse Model of Lung Adenocarcinoma

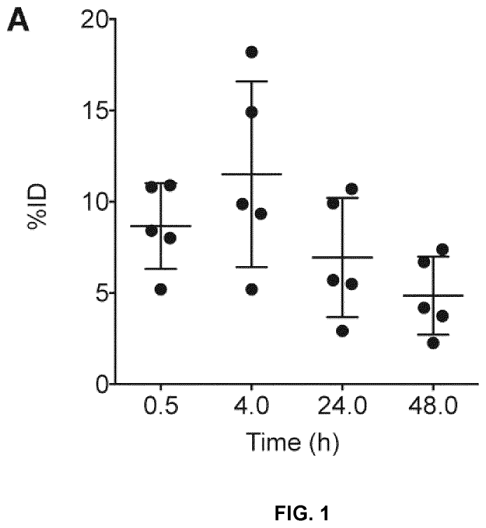

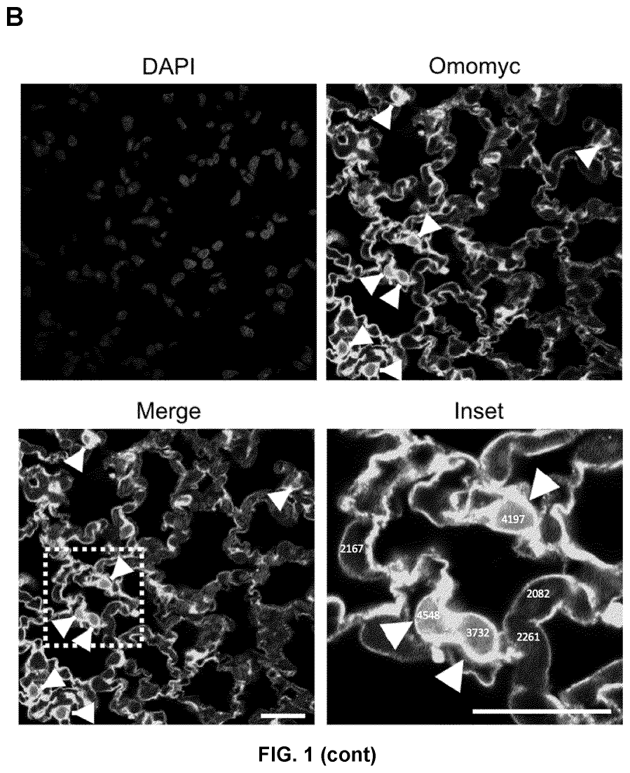

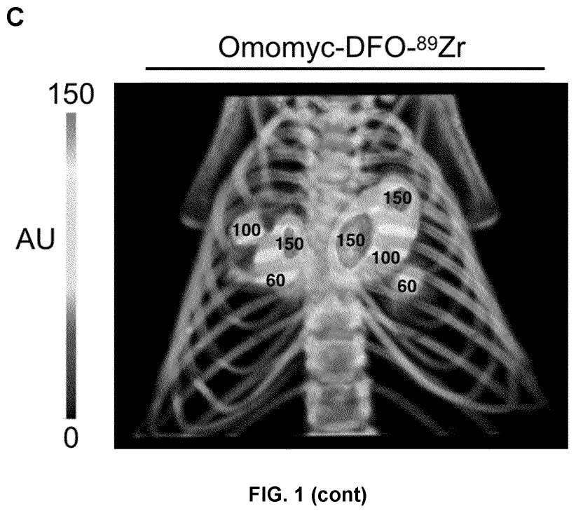

In order to evaluate the potential diagnostic utility of the Omomyc mini-protein in vivo, the inventors first analyzed its tissue distribution following intranasal administration (i.n.), a technique previously shown to enable direct pulmonary delivery of macromolecule formulations in mice. The inventors first covalently attached a deferoxamin-maleimide (DFO) group to Omomyc, and radiolabeled this with ⁸⁹Zr, then measured biodistribution and pharmacokinetic properties in healthy mice by ex vivo radiocounting (FIG. 1A). On average, 8% of the Omomyc-DFO-⁸⁹Zr dose (2.37 mg/kg) readily (within 30 minutes) reached the lungs following i.n. administration, and persisted there for more than 48 hours (FIG. 1A). Immunofluorescence using a specific anti-Omomyc antibody confirmed the detection (and partial nuclear localization) of unlabeled Omomyc mini-protein within the pulmonary epithelium of the treated mice at 4 hours post i.n. instillation (FIG. 1B). To confirm that this method was applicable to mice bearing lung adenocarcinoma, the inventors repeated the procedure using the well-characterized KRas^{LSL-G12D/+}-induced lung adenocarcinoma mouse model (E. L. Jackson. 2001. Genes & Development, 15:3243-3248). The mPET/mCT imaging of the mice revealed that, 24 hours after i.n. instillation, Omomyc-DFO-⁸⁹Zr localized mainly in the lung tumors (FIG. 1C). While the exact cause for this preferential tumor retention is unknown, it could be related to the altered tumor vasculature or metabolism typically observed in cancer.

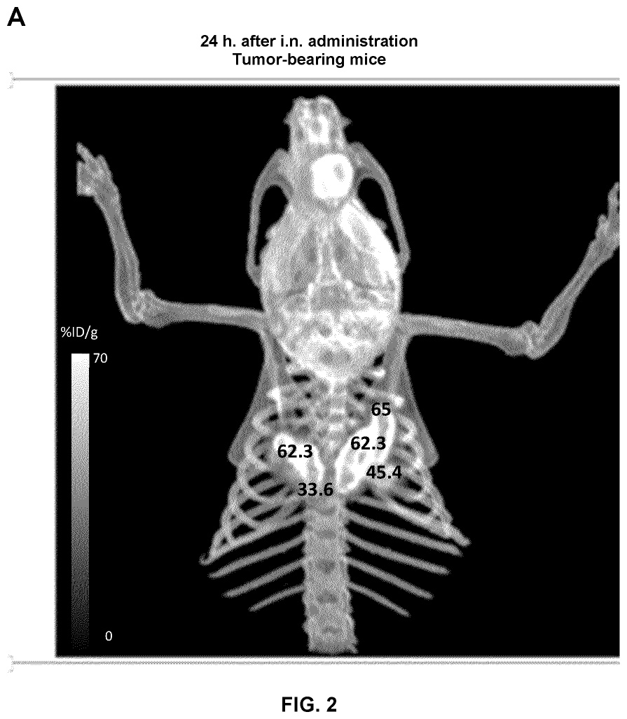

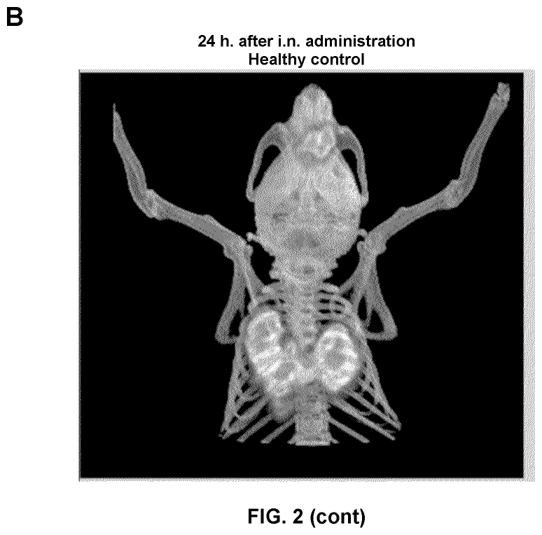

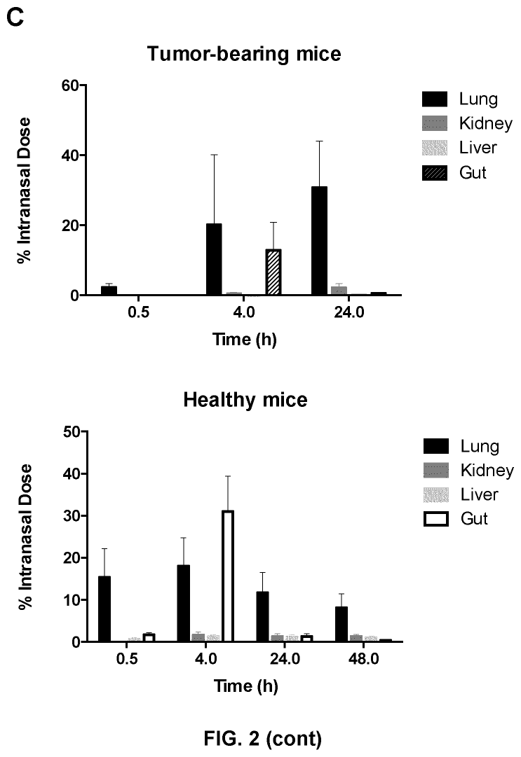

In a further experiment, 2 mg/kg of Omomyc-DFO-⁸⁹Zr were administered intranasally to a tumor-bearing Kras^{G12D} mice 18 weeks after AdCre induction. Images were obtained 24 hours after intranasal administration (FIG. 2A) where we can see that in the tumor-bearing mice the peptide present at 24 hours colocalizes with the tumors (FIG. 2A), whereas in the tumor-free mice, the lung distribution at the same time point is more diffuse throughout the lung (FIG. 2B). Numbers in FIG. 2A are indicative of the concentration of Omomyc-DFO-⁸⁹Zr, wherein higher numbers correspond to higher concentration of the conjugate. FIG. 2B shows an overexposed image of the diffuse and unspecific labeling of the lungs of the healthy controls, therefore, quantification is not displayed. Also, the total amount distributed to the lung is superior for the tumor-bearing mice compared to the healthy mice, although there is a high variability across individuals (FIG. 2C).

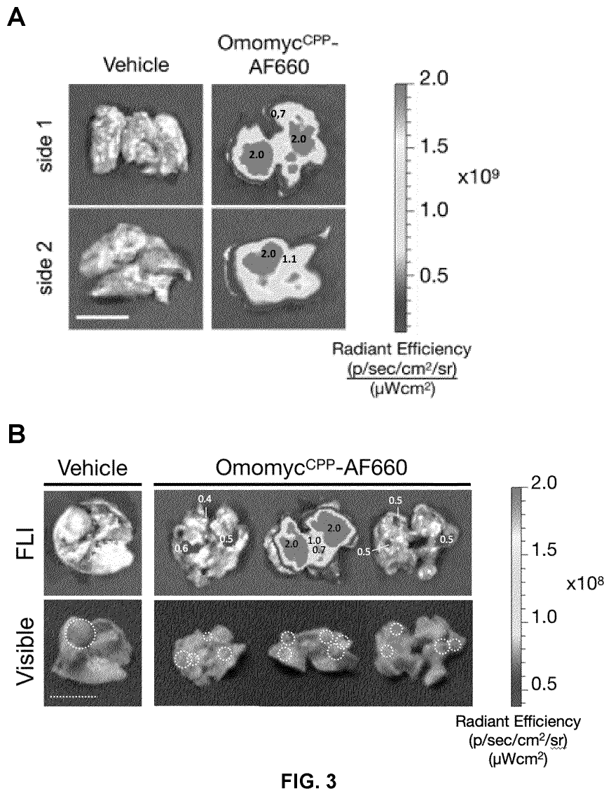

The same experiment was carried out with a different label (a fluorescent probe AF660 was used this time instead of the radioactive labelling) which allowed ex vivo imaging by IVIS Technology®. 4 h (FIG. 3A) after a single treatment with 1.4 mg/kg OmomycCPP-AF660, the fluorescent signal was detected in the lungs of mice. 24 h after intranasal administration, while most of OmomycCPP-AF660 had been washed out from normal tissues, it remained observable specifically in lung tumors (FIG. 3B). There again, the fluorescence signal colocalizes with the tumors following intranasal administration to mice (FIG. 3).

### Example 2. Biodistribution after Intravenous Administration of Omomyc in Mice Bearing Lung Tumors

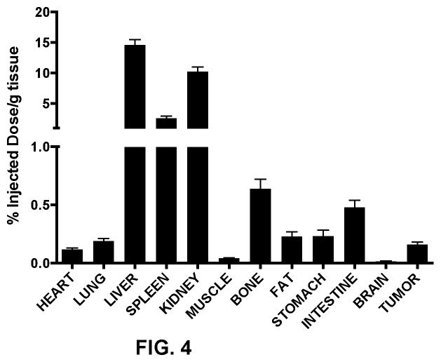

Five lung-bearing mice were intravenously administered with 29.1 mg/kg of Omomyc-DFO and PET/CT imaging was performed 72 h post-injection. As it is shown in FIG. 4, there is no significant Omomyc uptake increase in the lung compared to other groups. This result demonstrates the specificity of the Omomyc as a lung tumor tracer when administered directly to the lung.

1-23. (canceled)

24. A method for detecting or imaging lung cancer cells in a subject comprising:
i) administering by a pulmonary route a conjugate comprising a polypeptide comprising the sequence SEQ ID NO: 1 or a functionally equivalent variant thereof, and a detectable label;
ii) waiting a sufficient time to allow for the conjugate to enter into the proliferating lung cells; and
iii) detecting the proliferating lung cells by applying an imaging technique to the subject to detect the site of accumulation of the conjugate in the subject, thereby detecting or imaging the site of proliferation.

25. A diagnostic method for the detection of a lung tumour in a subject comprising:
i) detecting or imaging lung cancer cells in a subject by the method according to claim 24, and further
ii) identifying a lung tumour if a specific label is detected.

26. The method according to claim 25, wherein the functionally equivalent variant of SEQ ID NO: 1 is selected from the group consisting of SEQ ID NO: 4, SEQ ID NO: 5, SEQ ID NO;
6, SEQ ID NO: 7, SEQ ID NO: 8, SEQ ID NO: 9, and SEQ ID NO: 10.

27. The method according to claim 25, wherein the detectable label is selected from the group consisting of ¹⁸F, ³²P ³³P, ⁴⁵Ti, ⁴⁷Sc, ⁵²Fe, ⁵⁹Fe, ⁶²Cu, ⁶⁴Cu, ⁶⁷Ga, ⁶⁸Ga, ⁷⁵Sc, ⁷⁷As, ⁸⁶Y, ⁸⁹Sr, ⁸⁹Zr, ⁹⁴Tc, ⁹⁴Tc, ⁹⁹mTc, ⁹⁹Mo, ¹⁰⁵Pd, ¹⁵Rh, ¹¹¹Ag, ¹²³I, ¹²⁴I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁸⁶Re, ¹⁸⁹Re, ¹⁹⁴Ir. ¹⁹⁸Au, ¹⁹⁹Au, ²¹¹Pb, ²¹²Bi, ²¹²Pb, ²²³Ra and ²²⁵Ac and ⁸⁹Zr.

28. The method according to claim 25, wherein the imaging technique is mPET/mCT imaging.

29. The method according to claim 25, wherein the lung tumour is a primary tumour selected from small cell lung cancer (SCLC) and non-small cell lung cancer (NSCLC), or is a cancer metastasis.

30. The method according to claim 25, wherein the lung tumour is adenocarcinoma.

31. The method according to claim 30, wherein the adenocarcinoma is a KRAS-mutant adenocarcinoma.

32. The method according to claim 25, wherein the detection of the site of accumulation of the conjugate is carried out between 30 minutes and 96 hours after the administration of the conjugate to a subject in need thereof.

33. The method according to claim 25, wherein the administration by pulmonary route is carried out by nasal instillation, nasal inhalation or oral inhalation.

34. The method according to claim 25, wherein the dose of the conjugate ranges from 0.04 to 0.8 mg/Kg, or from 1.48 to 30 mg/m².

35. The method according to claim 25, further comprising administering the conjugate at least one more time for monitoring the progression of the lung tumour or the effect of a therapy administered to the subject.

36. The method according to claim 25, wherein said method is carried out by using a kit comprising:
(i) a conjugate comprising a polypeptide comprising the sequence SEQ ID NO: 1 or a functionally equivalent variant thereof, and a detectable label;
(ii) a device for the pulmonary administration of the conjugate of item (i); and
(iii) means for packaging items (i) and (ii).

37. Kit for the diagnosis of lung cancer comprising:
(i) a conjugate comprising a polypeptide comprising the sequence SEQ ID NO: 1 or a functionally equivalent variant thereof, and a detectable label;
(ii) a device for the pulmonary administration of the conjugate of item (i); and
(iii) means for packaging items (i) and (ii).

38. A conjugate comprising:
i) a polypeptide comprising the sequence SEQ ID NO: 1 or a functionally equivalent variant thereof, and
ii) a detectable label selected from the group consisting of a contrast agent or an imaging agent.

39. A method for treating a subject having lung cancer wherein said method comprises:
(i) selecting a subject as suffering from lung cancer by a method according to claim 25, and
(ii) administering to the subject identified with lung cancer a treatment selected from surgical removal of the lung tumor and/or administration of a chemotherapy and/or a radiotherapy.

40. A method for monitoring the progression of lung cancer in a subject, the method comprising:
(i) detecting or imaging lung cancer cells in the subject by a method according to claim 24, and
(ii) comparing the site of accumulation obtained in (i) with the site of accumulation obtained in a previous measure,
wherein a significant decrease or lack of change in the site of accumulation in comparison with the previous measure is indicative that the lung cancer is not progressing, or
wherein a significant increase in the site of accumulation in comparison with the previous measure is indicative that the lung cancer is progressing.

41. A method for monitoring the response to a therapy in a subject suffering from lung cancer, the method comprising:
(i) detecting or imaging lung cancer cells in the subject by a method according to claim 24, and
(ii) comparing the site of accumulation obtained in (i) with the site of accumulation obtained in a previous measure prior to administration of the therapy,
wherein a significant decrease or lack of change in the site of accumulation in comparison with the previous measure is indicative that the therapy administered to the subject is efficacious, or
wherein a significant increase in the site of accumulation in comparison with the previous measure is indicative that the therapy administered to the subject is inefficacious.

42. A method for treating a subject suffering from lung cancer, comprising administering a treatment selected from surgical removal of the lung tumor and/or administration of chemotherapy and/or radiotherapy, wherein the subject is identified by the diagnosis method of claim 25.

43. A method for treating a subject suffering from lung cancer, comprising administering a treatment selected from surgical removal of the lung tumor and/or administration of chemotherapy and/or radiotherapy, wherein the progression of lung cancer in the subject is assessed by the monitoring method of claim 40.
